SEQUENCE LISTING (31 sequences)

SEQ ID NO: 1 - protein, 153 aa, Homo sapiens [sequence omitted]

SEQ ID NO: 2 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 3 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 4 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 146 aa, Homo sapiens [sequence omitted]

SEQ ID NO: 7 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 8 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 9 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 10 - DNA, 562 bp, Homo sapiens
ctggaattgaggctgagccaaagaccccagggccgtctcagtctcataaaaggggatcaggcaggaggagtttgggagaaacctgagaagggcctgatttgcagcatcatgatgggcctctccttggcctctgctgtgctcctggcctccctcctgagtctccaccttggaactgccacacgtgggagtgacatatccaagacctgctgcttccaatacagccacaagccccttccctggacctgggtgcgaagctatgaattcaccagtaacagctgctcccagcgggctgtgatattcactaccaaaagaggcaagaaagtctgtacccatccaaggaaaaaatgggtgcaaaaatacatttctttactgaaaactccgaaacaattgtgactcagctgaattttcatccgaggacgcttggaccccgctcttggctctgcagccctctggggagcctgcggaatcttttctgaaggctacatggacccgctggggaggagagggtgtttcctcccagagttactttaataaaggttgttcatagagttgacttgttcat

SEQ ID NO: 11 - DNA, 3360 bp, Homo sapiens
gcgccagcttggagagccagccccatcggggttccccgccgccggaagcggaaatagcaccgggcgccgccacagtagctgtaactgccaccgcgatgccgaaggcgcccaagcagcagccgccggagcccgagtggatcggggacggagagagcacgagcccatcagacaaagtggtgaagaaagggaagaaggacaagaagatcaaaaaaacgttctttgaagagctggcagtagaagataaacaggctggggaagaagagaaagtgctcaaggagaaggagcagcagcagcagcaacagcaacagcagcaaaaaaaaaagcgagatacccgaaaaggcaggcggaagaaggatgtggatgatgatggagaagagaaagagctcatggagcgtcttaagaagctctcagtgccaaccagtgatgaggaggatgaagtacccgccccaaaaccccgcggagggaagaaaaccaagggtggtaatgtttttgcagccctgattcaggatcagagtgaggaagaggaggaggaagaaaaacatcctcctaagcctgccaagccggagaagaatcggatcaataaggccgtatctgaggaacagcagcctgcactcaagggcaaaaagggaaaggaagagaagtcaaaagggaaggctaagcctcaaaataaattcgctgctctggacaatgaagaggaggataaagaagaagaaattataaaggaaaaggagcctcccaaacaagggaaggagaaggccaagaaggcagagcagatggagtatgagcgccaagtggcttcattaaaagcagccaatgcagctgaaaatgacttctccgtgtcccaggcggagatgtcctcccgccaagccatgttagaaaatgcatctgacatcaagctggagaagttcagcatctccgctcatggcaaggagctgttcgtcaatgcagacctgtacattgtagccggccgccgctacgggctggtaggacccaatggcaagggcaagaccacactcctcaagcacattgccaaccgagccctgagcatccctcccaacattgatgtgttgctgtgtgagcaggaggtggtagcagatgagacaccagcagtccaggctgttcttcgagctgacaccaagcgattgaagctgctggaagaggagcggcggcttcagggacagctggaacaaggggatgacacagctgctgagaggctagagaaggtgtatgaggaattgcgggccactggggcggcagctgcagaggccaaagcacggcggatcctggctggcctgggctttgaccctgaaatgcagaatcgacccacacagaagttctcagggggctggcgcatgcgtgtctccctggccagggcactgttcatggagcccacactgctgatgctggatgagcccaccaaccacctggacctcaacgctgtcatctggcttaataactacctccagggctggcggaagaccttgctgatcgtctcccatgaccagggcttcttggatgatgtctgcactgatatcatccacctcgatgcccagcggctccactactataggggcaattacatgaccttcaaaaagatgtaccagcagaagcagaaagaactgctgaaacagtatgagaagcaagagaaaaagctgaaggagctgaaggcaggcgggaagtccaccaagcaggcggaaaaacaaacgaaggaagccctgactcggaagcagcagaaatgccgacggaaaaaccaagatgaggaatcccaggaggcccctgagctcctgaagcgccctaaggagtacactgtgcgcttcacttttccagaccccccaccactcagccctccagtgctgggtctgcatggtgtgacattcggctaccagggacagaaaccactctttaagaacttggattttggcatcgacatggattcaaggatttgcattgtgggccctaatggtgtggggaagagtacgctactcctgctgctgactggcaagctgacaccgacccatggggaaatgagaaagaaccaccggctgaaaattggcttcttcaaccagcagtatgcagagcagctgcgcatggaggagacgcccactgagtacctgcagcggggcttcaacctgccctaccaggatgcccgcaagtgcctgggccgcttcggcctggagagtcacgcccacaccatccagatctgcaaactctctggtggtcagaaggcgcgagttgtgtttgctgagctggcctgtcgggaacctgatgtcctcatcttggacgagccaaccaataacctggacatagagtctattgatgctctaggggaggccatcaatgaatacaagggtgctgtgatcgttgtcagccatgatgcccgactcatcacagaaaccaattgccagctgtgggtggtggaggagcagagtgttagccaaatcgatggtgactttgaagactacaagcgggaggtgttggaggccctgggtgaagtcatggtcagccggccccgagagtgagctttccttcccagaagtctcccgagagacatatttgtgtggcctagaagtcctctgtggtctcccctcctctgaagactgcctctggcctgcagctgacctggcaaccattcaggcacatgaaggtggagtgtgaccttgatgtgaccgggatcccactctgattgcatccatttctctgaaagacttgtttgttctgcttctcttcatataactgagctggccttatccttggcatccccctaaacaaacaagaggtgaccaccttattgtgaggttccatccagccaagtttatgtggcctattgtctcaggactctcatcactcagaagcctgcctctgatttaccctacagcttcaggcccagctgccccccagtctttgggtggtgctgttcttttctggtggatttaatgctgactcactggtacaaacagctgttgaagctcagagctggaggtgagcttctgaggcctttgccattatccagcccaagatttggtgcctgcagcctcttgtctggttgaggacttggggcaggaaaggaatgctgctgaacttgaatttccctttacaaggggaagaaataaaggaaaggagttgctgccgacctgtcactgtttggagattgatgggagttggaactgttctcagtcttgatttgctttattcagttttctagcagcttttaatagtcccctcttccccactaaatggatcttgtttgcagtcttgctgacagtgtttgctgtttaaggatcataggattcctttcccccaacccttcacgcaaggaaaaagcaaagtgattcataccttctatcttggaaaaaaaaaaaa

SEQ ID NO: 12 - DNA, 1119 bp, Homo sapiens
cacagagcccgggccgcaggcacctcctcgccagctcttccgctcctctcacagccgccagacccgcctgctgagccccatggcccgcgctgctctctccgccgcccccagcaatccccggctcctgcgagtggcactgctgctcctgctcctggtagccgctggccggcgcgcagcaggagcgtccgtggccactgaactgcgctgccagtgcttgcagaccctgcagggaattcaccccaagaacatccaaagtgtgaacgtgaagtcccccggaccccactgcgcccaaaccgaagtcatagccacactcaagaatgggcggaaagcttgcctcaatcctgcatcccccatagttaagaaaatcatcgaaaagatgctgaacagtgacaaatccaactgaccagaagggaggaggaagctcactggtggctgttcctgaaggaggccctgcccttataggaacagaagaggaaagagagacacagctgcagaggccacctggattgtgcctaatgtgtttgagcatcgcttaggagaagtcttctatttatttatttattcattagttttgaagattctatgttaatattttaggtgtaaaataattaagggtatgattaactctacctgcacactgtcctattatattcattctttttgaaatgtcaaccccaagttagttcaatctggattcatatttaatttgaaggtagaatgttttcaaatgttctccagtcattatgttaatatttctgaggagcctgcaacatgccagccactgtgatagaggctggcggatccaagcaaatggccaatgagatcattgtgaaggcaggggaatgtatgtgcacatctgttttgtaactgtttagatgaatgtcagttgttatttattgaaatgatttcacagtgtgtggtcaacatttctcatgttgaaactttaagaactaaaatgttctaaatatcccttggacattttatgtctttcttgtaaggcatactgccttgtttaatggtagttttacagtgtttctggcttagaacaaaggggcttaattattgatgttttcatagagaatataaaaataaagcacttatagaaaaaaaaaaaaaaaa

SEQ ID NO: 13 - DNA, 512 bp, Homo sapiens
tcccgagaagccaggaagcagtgagcccaggagtcctcggccagccctgcctgcccaccaggaggatgaaggtctccgtggctgccctctcctgcctcatgcttgttactgcccttggatcccaggcccgggtcacaaaagatgcagagacagagttcatgatgtcaaagcttccattggaaaatccagtacttctggacatgctctggaggagaaagattggtcctcagatgaccctttctcatgctgcaggattccatgctactagtgctgactgctgcatctcctacaccccacgaagcatcccgtgttcactcctggagagttactttgaaacgaacagcgagtgctccaagccgggtgtcatcttcctcaccaagaaggggcgacgtttctgtgccaaccccagtgataagcaagttcaggtttgcatgagaatgctgaagctggacacacggatcaagaccaggaagaattgaacttgtcaaggtgaagggacacaagttgccagc

SEQ ID NO: 14 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 15 - DNA, 1718 bp, Homo sapiens
gagggtgcataagttctctagtagggtgatgatataaaaagccaccggagcactccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 16 - DNA, 1351 bp, Homo sapiens
gtgatggagagcaccagcaaagccttagggcccatccctggcctcctgttacccacagaggggtaggcccttggctctcttccactatgacgtcagcttccattcttcctttcttatagacaattttccatttcaaggaaatcagagcccttaatagttcagtgaggtcactttgctgagcacaatcccatacccttcagcctctgctccacagagcctaagcaaaagatagaaactcacaacttccttgttttgttatctggaaattatcccaggatctggtgcttactcagcatattcaaggaaggtcttacttcattcttccttgattgtgaccatgcccaggctctctgctccctataaaaggcaggcagagccaccgaggagcagagaggttgagaacaacccagaaaccttcacctctcatgctgaagctcacacccttgccctccaagatgaaggtttctgcagcgcttctgtgcctgctgctcatggcagccactttcagccctcagggacttgctcagccagattcagtttccattccaatcacctgctgctttaacgtgatcaataggaaaattcctatccagaggctggagagctacacaagaatcaccaacatccaatgtcccaaggaagctgtgatcttcaagaccaaacggggcaaggaggtctgtgctgaccccaaggagagatgggtcagggattccatgaagcatctggaccaaatatttcaaaatctgaagccatgagccttcatacatggactgagagtcagagcttgaagaaaagcttatttattttccccaacctcccccaggtgcagtgtgacattattttattataacatccacaaagagattatttttaaataatttaaagcataatatttcttaaaaagtatttaattatatttaagttgttgatgttttaactctatctgtcatacatcctagtgaatgtaaaatgcaaaatcctggtgatgtgttttttgtttttgttttcctgtgagctcaactaagttcacggcaaaatgtcattgttctccctcctacctgtctgtagtgttgtggggtcctcccatggatcatcaaggtgaaacactttggtattctttggcaatcagtgctcctgtaagtcaaatgtgtgctttgtactgctgttgttgaaattgatgttactgtatataactatggaattttgaaaaaaaatttcaaaaagaaaaaaatatatataatttaaaactaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 791 bp, Homo sapiens
gccaggtgctgagacaaccacactatgagaggcactccaggagacgctgatggtggaggaagggccgtctatcaatcaatgtgtaaacctattactgggactattaatgatttgaatcagcaagtgtggacccttcagggtcagaaccttgtggcagttccacgaagtgacagtgtgaccccagtcactgttgctgttatcacatgcaagtatccagaggctcttgagcaaggcagaggggatcccatttatttgggaatccagaatccagaaatgtgtttgtattgtgagaaggttggagaacagcccacattgcagctaaaagagcagaagatcatggatctgtatggccaacccgagcccgtgaaacccttccttttctaccgtgccaagactggtaggacctccacccttgagtctgtggccttcccggactggttcattgcctcctccaagagagaccagcccatcattctgacttcagaacttgggaagtcatacaacactgcctttgaattaaatataaatgactgaactcagcctagaggtggcagcttggtctttgtcttaaagtttctggttcccaatgtgttttcgtctacattttcttagtgtcattttcacgctggtgctgagacaggggcaaggctgctgttatcatctcattttataatgaagaagaagcaattacttcatagcaactgaagaacaggatgtggcctcagaagcaggagagctgggtggtataaggctgtcctctcaagctggtgctgtgtaggccacaaggcatc

SEQ ID NO: 18 - DNA, 1834 bp, Homo sapiens
actttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaa

SEQ ID NO: 19 - DNA, 1234 bp, Homo sapiens
gagctccgggaatttccctggcccgggactccgggctttccagccccaaccatgcataaaaggggttcgccgttctcggagagccacagagcccgggccacaggcagctccttgccagctctcctcctcgcacagccgctcgaaccgcctgctgagccccatggcccgcgccacgctctccgccgcccccagcaatccccggctcctgcgggtggcgctgctgctcctgctcctggtggccgccagccggcgcgcagcaggagcgcccctggccactgaactgcgctgccagtgcttgcagaccctgcagggaattcacctcaagaacatccaaagtgtgaaggtgaagtcccccggaccccactgcgcccaaaccgaagtcatagccacactcaagaatgggcagaaagcttgtctcaaccccgcatcgcccatggttaagaaaatcatcgaaaagatgctgaaaaatggcaaatccaactgaccagaaggaaggaggaagcttattggtggctgttcctgaaggaggccctgcccttacaggaacagaagaggaaagagagacacagctgcagaggccacctggattgcgcctaatgtgtttgagcatcacttaggagaagtcttctatttatttatttatttatttatttgtttgttttagaagattctatgttaatattttatgtgtaaaataaggttatgattgaatctacttgcacactctcccattatatttattgtttattttaggtcaaacccaagttagttcaatcctgattcatatttaatttgaagatagaaggtttgcagatattctctagtcatttgttaatatttcttcgtgatgacatatcacatgtcagccactgtgatagaggctgaggaatccaagaaaatggccagtgagatcaatgtgacggcagggaaatgtatgtgtgtctattttgtaactgtaaagatgaatgtcagttgttatttattgaaatgatttcacagtgtgtggtcaacatttctcatgttgaagctttaagaactaaaatgttctaaatatcccttggacattttatgtctttcttgtaaggcatactgccttgtttaatgttaattatgcagtgtttccctctgtgttagagcagagaggtttcgatatttattgatgttttcacaaagaacaggaaaataaaatatttaaaaatataaaaaaaaaaaaaaaaaaaaaaaaaaaaaaaaa

SEQ ID NO: 20 - DNA, 1166 bp, Homo sapiens
gctccgggaatttccctggcccggccgctccgggctttccagtctcaaccatgcataaaaagggttcgccgatcttggggagccacacagcccgggtcgcaggcacctccccgccagctctcccgcttctcgcacagcttcccgacgcgtctgctgagccccatggcccacgccacgctctccgccgcccccagcaatccccggctcctgcgggtggcgctgctgctcctgctcctggtggccgccagccggcgcgcagcaggagcgtccgtggtcactgaactgcgctgccagtgcttgcagacactgcagggaattcacctcaagaacatccaaagtgtgaatgtaaggtcccccggaccccactgcgcccaaaccgaagtcatagccacactcaagaatgggaagaaagcttgtctcaaccccgcatcccccatggttcagaaaatcatcgaaaagatactgaacaaggggagcaccaactgacaggagagaagtaagaagcttatcagcgtatcattgacacttcctgcagggtggtccctgcccttaccagagctgaaaatgaaaaagagaacagcagctttctagggacagctggaaaggacttaatgtgtttgactatttcttacgagggttctacttatttatgtatttatttttgaaagcttgtattttaatattttacatgctgttatttaaagatgtgagtgtgtttcatcaaacatagctcagtcctgattatttaattggaatatgatgggttttaaatgtgtcattaaactaatatttagtgggagaccataatgtgtcagccaccttgataaatgacagggtggggaactggagggtggggggattgaaatgcaagcaattagtggatcactgttagggtaagggaatgtatgtacacatctattttttatactttttttttaaaaaaagaatgtcagttgttatttattcaaattatctcacattatgtgttcaacatttttatgctgaagtttcccttagacattttatgtcttgcttgtagggcataatgccttgtttaatgtccattctgcagcgtttctctttcccttggaaaagagaatttatcattactgttacatttgtacaaatgacatgataataaaagttttatgaaaaaaaaaaaaaaa

SEQ ID NO: 21 - DNA, 3686 bp, Homo sapiens
cttcagatagattatatctggagtgaagaatcctgccacctatgtatctggcatagtattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccgggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataaaaaaaaaaaaaaaaaaaaaaaaaaa

SEQ ID NO: 22 - DNA, 5821 bp, Homo sapiens
aaacaaaacagaaatgcaacgctttagagtacccacggaaaacttgtttaccttgtcaccatgagtaaaagttaattcccactcctgaagagagcaaaccaactctgaaagagagtgaaaatgcagacaagacagttatcagataatggctatctggacgagagattctttcgtttgacagcagtttggttgttgggagttccagttcagctcctgcacagttgctctgtacaaatcctcctccatatttgcttagagaaaacgtgttgccatcccatcatgaaggaagctgcctgagagtttttaaccattacagccgtgatgatgaaagagtgaagaaccgcctctaagttaaaaagtgcacccagagataaggttcgttctcaatgccctgccgctgcttctcatcgcatggccaccgcatttctcaggccaggcacattgagcattggtcctgtgcctgacgctatgctagatgctggggttgcagccacgagcatagacacgacagacacggtcctcgccatcttctgttgagtactggtcggaacaagaggatcgtctgtagacaggctacagattgttttagattgaagtttcctgtcatgttcactcatctttaaatcctcatagtgaaaaaggatatgatcatcgtggcgcatgtattactcatccttttgggggccactgagatactgcaagctgacttacttcctgatgaaaagatttcacttctcccacctgtcaatttcaccattaaagttactggtttggctcaagttcttttacaatggaaaccaaatcctgatcaagagcaaaggaatgttaatctagaatatcaagtgaaaataaacgctccaaaagaagatgactatgaaaccagaatcactgaaagcaaatgtgtaaccatcctccacaaaggcttttcagcaagtgtgcggaccatcctgcagaacgaccactcactactggccagcagctgggcttctgctgaacttcatgccccaccagggtctcctggaacctcaattgtgaatttaacttgcaccacaaacactacagaagacaattattcacgtttaaggtcataccaagtttcccttcactgcacctggcttgttggcacagatgcccctgaggacacgcagtattttctctactataggtatggctcttggactgaagaatgccaagaatacagcaaagacacactggggagaaatatcgcatgctggtttcccaggacttttatcctcagcaaagggcgtgactggcttgcggtgcttgttaacggctccagcaagcactctgctatcaggccctttgatcagctgtttgcccttcacgccattgatcaaataaatcctccactgaatgtcacagcagagattgaaggaactcgtctctctatccaatgggagaaaccagtgtctgcttttccaatccattgctttgattatgaagtaaaaatacacaatacaaggaatggatatttgcagatagaaaaattgatgaccaatgcattcatctcaataattgatgatctttctaagtacgatgttcaagtgagagcagcagtgagctccatgtgcagagaggcagggctctggagtgagtggagccaacctatttatgtgggaaatgatgaacacaagcccttgagagagtggtttgtcattgtgattatggcaaccatctgcttcatcttgttaattctctcgcttatctgtaaaatatgtcatttatggatcaagttgtttccaccaattccagcaccaaaaagtaatatcaaagatctctttgtaaccactaactatgagaaagctgggtccagtgagacggaaattgaagtcatctgttatatagagaagcctggagttgagaccctggaggattctgtgttttgactgtcactttggcatcctctgatgaactcacacatgcctcagtgcctcagtgaaaagaacagggatgctggctcttggctaagaggtgttcagaatttaggcaacactcaatttacctgcgaagcaatacacccagacacaccagtcttgtatctcttaaaagtatggatgcttcatccaaatcgcctcacctacagcagggaagttgactcatccaagcattttgccatgttttttctccccatgccgtacagggtagcacctcctcacctgccaatctttgcaatttgcttgactcacctcagacttttcattcacaacagacagcttttaaggctaacgtccagctgtatttacttctggctgtgcccgtttggctgtttaagctgccaattgtagcactcagctaccatctgaggaagaaagcattttgcatcagcctggagtgaaccatgaacttggattcaagactgtcttttctatagcaagtgagagccacaaattcctcacccccctacattctagaatgatctttttctaggtagattgtgtatgtgtgtgtatgagagagagagagagagagagagagagagaaattatctcaagctccagaggcctgatccaggatacatcatttgaaaccaactaatttaaaagcataatagagctaatatatcaccccatatcaaaagagacgacagattttgttcaaatattatattcttgaaggaagccttaatattctggaaatgtgctgaggaagtcattgagcataattcaatcatgaaagggaccactgaatcaagaatgaactttctgaagagatgtttgctgccaaagacttcagaacaacttgttggtcaagtaatgccagatcaatcctagtctctcaagtttggaaaagtttctacaaggccattccatcaaatatcccaatatacacagaaattcttcttcctaacatcttcatatatgagtctgcatccagattggaggcctttgtggtgggtggtggactgcaggtggaaccaagcacaggtgtgccctgggcagaggtgggcaagaggaaggaacccaaacagtgctgattccacatggcccttggtcaagaagctgaaggcctagaaaggaggtaacagtgcccagaatccccacctggcacactctttaaatgctttctggaggtgttttagtcagttttgatccatcggtggggacacactgactaccatatatttccaggtttgtttgttttcttaactaacctgtaattatccactgtaactagcagagggttgaaccctgggtaataggcacagaaacatcttgaatcttgacaagcctgcactgtcttatagtaatctgtattactgtcccattttatagttgcagttactaaggttcagagaagttgataggaaatggcatagtgaggagttgaataccaggctcaggtaaaagtgaccccagaaatgtgcagaggagtgtcagggaaagctgaggcctgcttaggggtgtggctgagaaggcaacacccatggaggtgctgcaatagttggcaaaatgtttaaaatgtttggtcactcaaatgattcataatctcaaacctgtttttgtttttttttttttttttttttgtggggtgggtggtggagacagtcttgttctgtctcccaggctagagtgtagtgatgtgatcttagctcactacaacctccctgtcctgggctcaagccatcctcctccctcaccctcccaagtagctgggactacaggtgcataacaccattcccagctcttttttgtatttttttttgtaaagctggggttttgccatgctgtctaggttcatctcgaactcctgggctcaagcgatacaccggcctcagccttccaaagtactgggattacaggcatgaaccaccattcctggccaggaaactgtatttgtaatgaacaccttaggtcagaatcagaggcgcctggagggctccttaagacacagaggagtgggctccattcttagagtctctaactgaataggtctgggtgaggtctgaaaagctgcgtttctaacaagttctcaggtgatgctcatgctgctggtctccaggacacactcggagaactgctgctccaggagagtcttatgaataccaaaatttgaaaatcagccgctgaaggaagatgtgacagaaaagtcctgtccccagaaaactgtccttttggggcggcttggagcaccctgtctgatctctttcaaaccacaacccagacagttggcattgtggaaactgcctctgcttaggaggcaggaacagaagcagggagctccccctccacaaatgtggccagctcttcagtgcttggtgtggtgcccacttcagactcctccctccactcagcagccatagatcacctgggctctgtggagctccttgccacacttccctgagctggtaccgttatctggcccttggtatatgtgtttcaggagcaacacaaagtagcccattggcctccttggtggattgggaactccttcctcccttcttgccctgtgcaactcatagtcattctttgggaacccgtaaccatcatcacttcctccatgaagccttcaaaagcagagttggttgatcccgcctctggattctcattctgggcctcatacccgtgttagagctactgcattgtagaatataccattaacagctgggaggcctcccctctagactgcggcttccaggaggatggagcttgtgtgaaatacgtctttttatctgcagtgtctaggacagtatctgacttaagaaaatataaatgtttcttaaaccctctgtcagccctgatacggcaccttgtatataggttagactgaacagtatttggcagagatcctcaaggcctacaataatgacagggggaggaagctgggtatctaatgagaatgcggttagttagaagcaaccaggagacacatgtgcctaacaactgggctactgcaacctggatgtttttgctgccaaagtgtctaaatggttatgtataaattgctgtgtaagaggcatttcagaagtggctcactgtgagtggtctatactgttctcaccagccttgtcatagcaagtcaaagagtttgaaattcacttaacagtaaacaccatcagcagcaggttgatgtaaggtaaagtcatgatattggctcccatctcttgccatctctcactaggacgagtgtagagagtcacttttctcctagagcctccacagccacgggatcagaacaaacatgcttttccaagatatccgttccaatagggatttttagcccaatttctttccttggcatttggaattcactgcgtgcagcatgggggagtgctggggagactggagggtgaaggtcacatttgccaacagcccgaccacctgagcctcattgaaatgcctcagacctcaagctattcatgcaggctggggaagaatccacgtgggcaaagaacatcctgattaaaaatccaaacgttttcatggtgagacttctatgtgtcaggaccatctgctggtggaattaagaagcctgaggtgctaatttgttaaagaaaaagtagatatctggaagtgagaacagagaactgaaaacaatctctgatggcacaagtaaaggtgaaaaacctaattagaataggacttggtgctagcctaaggactgtagctattcctgcttctacatccttcaaattgagtaccagggtgcaaattttttcaaaaccattccttcctttggaattttgtattgtactataagacaatcatgcttatctcctgttttgtaggtaatagatgtgaatataaatatgtttcagaatgagaaatattaaacataaatgacagtgaaaaaaaaaaaaaaa

SEQ ID NO: 23 - DNA, 542 bp, Homo sapiens
acagtggtgagctcttagcttcaccaggctcatcaaagctgctccaggaaggcccaagccagaccagaagacatgcagatcatcaccacagccctggtgtgcttgctgctagctgggatgtggccggaagatgtggacagcaagagcatgcaggtacccttctccagatgttgcttctcatttgcggagcaagagattcccctgagggcaatcctgtgttacagaaataccagctccatctgctccaatgagggcttaatattcaagctgaagagaggcaaagaggcctgcgccttggacacagttggatgggttcagaggcacagaaaaatgctgaggcactgcccgtcaaaaagaaaatgagcagatttctttccattgtgggctctggaaaccacatggcttcacctgtccccgaaactaccagccctacaccattccttctgccctgcttttgctaggtcacagaggatctgcttggtcttgataagctatgttgttgcactttaaacatttaaattatacaatcatcaacccccaac

SEQ ID NO: 24 - DNA, 591 bp, Homo sapiens
ccgctgtcaagatgcttctggccatggtccttacctctgccctgctcctgtgctccgtggcaggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatatgaagatgaaatattatttatcctatttattaaatttaaaaagctttctctttaagttgctacaatttaaaaatcaagtaagctactctaaatcagtatcagttgtgattatttgtttaacattgtatgtctttattttgaaataaat

SEQ ID NO: 25 - DNA, 1048 bp, Homo sapiens
gccaggtgtgcaggccgctccaagcccagcctgccccgctgccgccaccatgacgctcctccccggcctcctgtttctgacctggctgcacacatgcctggcccaccatgacccctccctcagggggcacccccacagtcacggtaccccacactgctactcggctgaggaactgcccctcggccaggcccccccacacctgctggctcgaggtgccaagtgggggcaggctttgcctgtagccctggtgtccagcctggaggcagcaagccacagggggaggcacgagaggccctcagctacgacccagtgcccggtgctgcggccggaggaggtgttggaggcagacacccaccagcgctccatctcaccctggagataccgtgtggacacggatgaggaccgctatccacagaagctggccttcgccgagtgcctgtgcagaggctgtatcgatgcacggacgggccgcgagacagctgcgctcaactccgtgcggctgctccagagcctgctggtgctgcgccgccggccctgctcccgcgacggctcggggctccccacacctggggcctttgccttccacaccgagttcatccacgtccccgtcggctgcacctgcgtgctgccccgttcagtgtgaccgccgaggccgtggggcccctagactggacacgtgtgctccccagagggcaccccctatttatgtgtatttattgttatttatatgcctcccccaacactacccttggggtctgggcattccccgtgtctggaggacagccccccactgttctcctcatctccagcctcagtagttgggggtagaaggagctcagcacctcttccagcccttaaagctgcagaaaaggtgtcacacggctgcctgtaccttggctccctgtcctgctcccggcttcccttaccctatcactggcctcaggcccccgcaggctgcctcttcccaacctccttggaagtacccctgtttcttaaacaattatttaagtgtacgtgtattattaaactgatgaacacatccccaaaa

SEQ ID NO: 26 - DNA, 3662 bp, Homo sapiens
gcgagctggtggttgaagctggttaaagaacagcctaggtattccagaagtgtttgaggatcccttccatgaaggaagagaggaaagtttttaagtaaacctcccactcccatgtgtcttcagctttcttttgcaaaggagaaaatccttgaagtttggtaaagaccgagttagtctatctctctttgcctatctcgagttgggctggggagaggaggagataggttcttttgtctttttctgtcttctcccttccccacttccttccctccagtccccactcactcacatgcacacactaaccttggagccgatgggattgagtgactggcacttgggaccacagagaaatgtcagagtgtttggttacagactcaaggaaacctctcattttagagtgctcatttggttttgagcaaaattttggactgtgaagcaaggcattggtgaagacaaaatggcctcgccggctgacagctgtatccagttcacccgccatgccagtgatgttcttctcaaccttaatcgtctccggagtcgagacatcttgactgatgttgtcattgttgtgagccgtgagcagtttagagcccataaaacggtcctcatggcctgcagtggcctgttctatagcatctttacagaccagttgaaatgcaaccttagtgtgatcaatctagatcctgagatcaaccctgagggattctgcatcctcctggacttcatgtacacatctcggctcaatttgcgggagggcaacatcatggctgtgatggccacggctatgtacctgcagatggagcatgttgtggacacttgccggaagtttattaaggccagtgaagcagagatggtttctgccatcaagcctcctcgtgaagagttcctcaacagccggatgctgatgccccaagacatcatggcctatcggggtcgtgaggtggtggagaacaacctgccactgaggagcgcccctgggtgtgagagcagagcctttgcccccagcctgtacagtggcctgtccacaccgccagcctcttattccatgtacagccacctccctgtcagcagcctcctcttctccgatgaggagtttcgggatgtccggatgcctgtggccaaccccttccccaaggagcgggcactcccatgtgatagtgccaggccagtccctggtgagtacagccggccgactttggaggtgtcccccaatgtgtgccacagcaatatctattcacccaaggaaacaatcccagaagaggcacgaagtgatatgcactacagtgtggctgagggcctcaaacctgctgccccctcagcccgaaatgccccctacttcccttgtgacaaggccagcaaagaagaagagagaccctcctcggaagatgagattgccctgcatttcgagccccccaatgcacccctgaaccggaagggtctggttagtccacagagcccccagaaatctgactgccagcccaactcgcccacagagtcctgcagcagtaagaatgcctgcatcctccaggcttctggctcccctccagccaagagccccactgaccccaaagcctgcaactggaagaaatacaagttcatcgtgctcaacagcctcaaccagaatgccaaaccagaggggcctgagcaggctgagctgggccgcctttccccacgagcctacacggccccacctgcctgccagccacccatggagcctgagaaccttgacctccagtccccaaccaagctgagtgccagcggggaggactccaccatcccacaagccagccggctcaataacatcgttaacaggtccatgacgggctctccccgcagcagcagcgagagccactcaccactctacatgcaccccccgaagtgcacgtcctgcggctctcagtccccacagcatgcagagatgtgcctccacaccgctggccccacgttccctgaggagatgggagagacccagtctgagtactcagattctagctgtgagaacggggccttcttctgcaatgagtgtgactgccgcttctctgaggaggcctcactcaagaggcacacgctgcagacccacagtgacaaaccctacaagtgtgaccgctgccaggcctccttccgctacaagggcaacctcgccagccacaagaccgtccataccggtgagaaaccctatcgttgcaacatctgtggggcccagttcaaccggccagccaacctgaaaacccacactcgaattcactctggagagaagccctacaaatgcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccactagtgcaaatcatagctgggggttgggggtggtgggggtcggggcctgggggactgggagccgcagcagctccccctcccccactgccataaaacattaagaaaatcatattgcttcttctcctatgtgtaaggtgaaccatgtcagcaaaaagcaaaatcattttatatgtcaaagcaggggagtatgcaaaagttctgacttgactttagtctgcaaaatgaggaatgtatatgttttgtgggaacagatgtttcttttgtatgtaaatgtgcattcttttaaaagacaagacttcagtatgttgtcaaagagagggctttaatttttttaaccaaaggtgaaggaatatatggcagagttgtaaatatataaatatatatatatataaaataaatatatataaacctaaaaaagatatattaaaaatataaaactgcgttaaaggctcgattttgtatctgcaggcagacacggatctgagaatctttattgagaaagagcacttaagagaatattttaagtattgcatctgtataagtaagaaaatattttgtctaaaatgcctcagtgtatttgtatttttttgcaagtgaaggtttacaatttacaaagtgtgtattaaaaaaaacaaaaagaacaaaaaaatctgcagaaggaaaaatgtgtaattttgttctagttttcagtttgtatatacccgtacaacgtgtcctcacggtgccttttttcacggaagttttcaatgatgggcgagcgtgcaccatccctttttgaagtgtaggcagacacagggacttgaagttgttactaactaaactctctttgggaatgtttgtctcatcccattctgcgtcatgcttgtgttataactactccggagacagggtttggctgtgtctaaactgcattaccgcgttgtaaaatatagctgtacaaatataagaataaaatgttgaaaagtcaaactggaaa

SEQ ID NO: 27 - DNA, 1282 bp, Homo sapiens
aagccacccagcctatgcatccgctcctcaatcctctcctgttggcactgggcctcatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaactgaaacttcgaaagcatcattatttgcagagacaggacctgactattgaagttgcagattcatttttctttctgatgtcaaaaatgtcttgggtaggcgggaaggagggttagggaggggtaaaattccttagcttagacctcagcctgtgctgcccgtcttcagcctagccgacctcagccttccccttgcccagggctcagcctggtgggcctcctctgtccagggccctgagctcggtggacccagggatgacatgtccctacacccctcccctgccctagagcacactgtagcattacagtgggtgccccccttgccagacatgtggtgggacagggacccacttcacacacaggcaactgaggcagacagcagctcaggcacacttcttcttggtcttatttattattgtgtgttatttaaatgagtgtgtttgtcaccgttggggattggggaagactgtggctgctagcacttggagccaagggttcagagactcagggccccagcactaaagcagtggacaccaggagtccctggtaataagtactgtgtacagaattctgctacctcactggggtcctggggcctcggagcctcatccgaggcagggtcaggagaggggcagaacagccgctcctgtctgccagccagcagccagctctcagccaacgagtaatttattgtttttccttgtatttaaatattaaatatgttagcaaagagttaatatatagaagggtaccttgaacactgggggaggggacattgaacaagttgtttcattgactatcaaactgaagccagaaataaagttggtgacagat

SEQ ID NO: 28 - DNA, 851 bp, Homo sapiens
agaatataacagcactcccaaagaactgggtactcaacactgagcagatctgttctttgagctaaaaaccatgtgctgtaccaagagtttgctcctggctgctttgatgtcagtgctgctactccacctctgcggcgaatcagaagcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatgtaaaaactgtggcttttctggaatggaattggacatagcccaagaacagaaagaaccttgctggggttggaggtttcacttgcacatcatggagggtttagtgcttatctaatttgtgcctcactggacttgtccaattaatgaagttgattcatattgcatcatagtttgctttgtttaagcatcacattaaagttaaactgtattttatgttatttatagctgtaggttttctgtgtttagctatttaatactaattttccataagctattttggtttagtgcaaagtataaaattatatttgggggggaataagattatatggactttcttgcaagcaacaagctattttttaaaaaaaactatttaacattcttttgtttatattgttttgtctcctaaattgttgtaattgcattataaaataagaaaaatattaataagacaaatattgaaaataaagaaacaaaaagttcttctgttaaaaaaaaa

SEQ ID NO: 29 - DNA, 1794 bp, Homo sapiens
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 30 - DNA, 477 bp, Homo sapiens
atggaaaaagcattgaaaattgacacacctcagcaggggagcattcaggatatcaatcatcgggtgtgggttcttcaggaccagacgctcatagcagtcccgaggaaggaccgtatgtctccagtcactattgccttaatctcatgccgacatgtggagacccttgagaaagacagagggaaccccatctacctgggcctgaatggactcaatctctgcctgatgtgtgctaaagtcggggaccagcccacactgcagctgaaggaaaaggatataatggatttgtacaaccaacccgagcctgtgaagtcctttctcttctaccacagccagagtggcaggaactccaccttcgagtctgtggctttccctggctggttcatcgctgtcagctctgaaggaggctgtcctctcatccttacccaagaactggggaaagccaacactactgactttgggttaactatgctgttttaa

SEQ ID NO: 31 - DNA, 1989 bp, Homo sapiens
aatgtgtgcgcgctgtggtatgggtgtgcaagtgtgcgaaggcggcgtgttgtgtgagcgagagggtagcggatgtgtgtgtgcgtgtgcgcgcgtggctccgggtgtgcgccgctgcgatagcgggtcctttcccggggcgggcgacgggcgggctgggaaggtctcctcccctcaccacattgagaaatctcagtgagtcaccgagtggttctgcatattaatgagctcgctcgctgcgagggcaggagcggatttaaaagaggccagggcgggcggagggaggctgtggagagagcgcggagacaagcgcagagcgcagcgcacggccacagacagccctgggcatccaccgacggcgcagccggagccagcagagccggaaggcgcgccccgggcagagaaagccgagcagagctgggtggcgtctccgggccgccgctccgacgggccagcgccctccccatgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatagggtgaaaaacctcagaagggaaaactccaaaccagttgggagacttgtgcaaaggactttgcagattaaaaaaaaaaaaaaaaaaaaaaaaaaaaaaaaaaaaaaaaaaagcctttctttctcacaggcataagacacaaattatatattgttatgaagcactttttaccaacggtcagtttttacattttatagctgcgtgcgaaaggcttccagatgggagacccatctctcttgtgctccagacttcatcacaggctgctttttatcaaaaaggggaaaactcatgcctttcctttttaaaaaatgcttttttgtatttgtccatacgtcactatacatctgagctttataagcgcccgggaggaacaatgagcttggtggacacatttcattgcagtgttgctccattcctagcttgggaagcttccgcttagaggtcctggcgcctcggcacagctgccacgggctctcctgggcttatggccggtcacagcctcagtgtgactccacagtggcccctgtagccgggcaagcaggagcaggtctctctgcatctgttctctgaggaactcaagtttggttgccagaaaaatgtgcttcattcccccctggttaatttttacacaccctaggaaacatttccaagatcctgtgatggcgagacaaatgatccttaaagaaggtgtggggtctttcccaacctgaggatttctgaaaggttcacaggttcaatatttaatgcttcagaagcatgtgaggttcccaacactgtcagcaaaaaccttaggagaaaacttaaaaatatatgaatacatgcgcaatacacagctacagacacacattctgttgacaagggaaaaccttcaaagcatgtttctttccctcaccacaacagaacatgcagtactaaagcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttatatgctatgtacatgtcagaaaccattagcattgcatgcaggtttcatattctttctaagatggaaagtaataaaatatatttgaaatgtaccaaaaaaaaaaaaaaaaaa

(19) United States
(12) Patent Application Publication
(10) Pub. No.: US 2013/0324435 A1
(43) Pub. Date: Dec. 5, 2013

(54) ESOPHAGEAL CYTOKINE EXPRESSION PROFILES IN EOSINOPHILIC ESOPHAGITIS

(71) Applicants: Marc E. Rothenberg, Cincinnati, OH (US); Carine Blanchard, Le Mont sur Lausanne (CH)

(72) Inventors: Marc E. Rothenberg, Cincinnati, OH (US); Carine Blanchard, Le Mont sur Lausanne (CH)

(73) Assignee: CHILDREN'S HOSPITAL MEDICAL CENTER, Cincinnati, OH (US)

(21) Appl. No.: 13/978,117

(22) PCT Filed: Jan. 6, 2012
(86) PCT No.: PCT/US2012/020556

§ 371 Date: Jul. 2, 2013

Related U.S. Application Data
(60) Provisional application No. 61/430,453, filed on Jan. 6, 2011.

(51) Int. Cl. C12Q 1/68

(52) U.S. Cl. CPC C12Q 1/6883
USPC 506/9; 435/6.11; 435/6.12; 506/16

(57) ABSTRACT

Methods and compositions disclosed herein generally relate to methods of providing or enhancing a diagnosis of eosinophilic esophagitis (EE). In particular, the invention relates to obtaining a sample from a patient having at least one indication of EE, then quantifying from the sample an amount of one or more cytokines associated with EE or an mRNA corresponding to the cytokine or its receptor, wherein an altered level of the cytokine or mRNA correlates with a positive diagnosis of EE. An EE diagnosis can then be provided or enhanced, based upon the quantifying step. The invention further relates to diagnostic kits, tests, and/or arrays that can be used to quantify the one or more cytokines associated with EE or an mRNA corresponding to the cytokine or its receptor.

### CROSS REFERENCE TO RELATED APPLICATION

The present application claims the benefit of priority under 35 U.S.C. §119(e) to U.S. Provisional Application No. 61/430,453, A STRIKING LOCAL ESOPHAGEAL CYTOKINE EXPRESSION PROFILE IN EOSINOPHILIC ESOPHAGITIS, filed on Jan. 6, 2011, which is currently co-pending herewith and which is incorporated by reference in its entirety.

### STATEMENT REGARDING FEDERALLY-SPONSORED RESEARCH

This invention was made with U.S. Government support. This work was supported in part by the Pilot and Feasibility Program PHS Grant P30 DK0789392 and by NIH grants AI079874-01, AI070235, AI045898, and DK076893. The U.S. Government could have certain rights in the subject matter hereof

### FIELD OF THE INVENTION

The invention disclosed herein generally relates to diagnosis, treatment, and/or management of eosinophilic esophagitis and/or diseases, disorders, and/or conditions arising therefrom and/or related thereto.

### BACKGROUND

All publications mentioned herein are incorporated by reference to the same extent as if each individual publication or patent application was specifically and individually indicated to be incorporated by reference. The following description includes information that can be useful in understanding the present subject matter. It is not an admission that any of the information provided herein is prior art or relevant to the presently claimed subject matter, or that any publication specifically or implicitly referenced is prior art.

Eosinophilic esophagitis (EE, also abbreviated EoE in some publications) is an emerging worldwide disease characterized by marked esophageal eosinophil infiltration (>15 eosinophils/high power field [hpf]) that is not responsive to acid suppressive therapy (see, e.g., Furuta, G. et al., Gastroenterology 133:1342-63 (2007); Assa'ad, A. et al. J Allergy Clin. Immunol. 119:731-8 (2007); Straumann, A. and Simon, H. J Allergy Clin. Immunol. 115:418-9 (2005)). EE symptoms mimic gastroesophageal reflux disease (GERD) and vary with age. Patients with EE can have gastrointestinal complains that typically include, but are not limited to, failure to thrive, vomiting, abdominal pain, dysphagia, and food impactions (see, e.g., Furuta, G. et al., Gastroenterology 133:1342-63 (2007); Liacouras, C. et al. J. Pediatr. Gastroenterol. Nutr. 45:370-91 (2007)).

EE diagnosis generally involves endoscopy, which is an invasive and inconvenient procedure. The endoscopy procedure is then commonly followed by biopsy analysis.

### SUMMARY OF THE INVENTION

Methods and compositions described herein are provided by way of example and should not in any way limit the scope of the invention.

Embodiments of the invention encompass methods of providing or enhancing a diagnosis of EE, including: obtaining a sample from a patient having at least one indication of EE; quantifying from the sample an amount of at least one analyte, wherein the analyte can be, for example, any of the cytokines listed in Table 1, any of the cytokines listed in Table 2, or an mRNA corresponding to any member of the group or its receptor, or the like, wherein an altered level of the at least one analyte correlates with a positive diagnosis of EE; and providing or enhancing a diagnosis of EE, based upon the quantifying step.

In some embodiments of the methods, the at least one analyte can be, for example, any of the cytokines listed in Table 1, or an mRNA corresponding to any member of the group or its receptor, or the like. In some embodiments, at least two analytes can be quantified; in others, at least four analytes can be quantified, and in others, all of the analytes in Table 1 can be quantified, and in others, all of the analytes in Table 2 can be quantified.

In some embodiments, the sample can include, for example, an esophageal biopsy, and/or esophageal mucosa, and/or include blood, and/or the like. Blood can include, for example, plasma, serum, whole blood, blood lysates, and the like.

In some embodiments, the indication of EE can include one or more of a gastrointestinal complaint, esophageal eosinophil infiltration, and the like. The gastrointestinal complaint can include, for example, one or more of: failure to thrive, vomiting, abdominal pain, dysphagia, food impaction, and the like.

In some embodiments, the diagnosis of EE can include classification as allergic, non-allergic, active, intermediate, or inactive EE, a variable degree of disease activity, or the like. In some embodiments, the EE diagnosis classification can be used to predict the patient's level of response to a selected therapy. In some embodiments, the selected therapy can include, for example, allergen removal, steroid treatment, dietary management, the use of proton pump inhibitors (PPIs), topical glucocorticoids, humanized antibodies against relevant cytokines, and small molecule inhibitors of an eosinophil and/or allergic disease activation pathway, or the like. In some embodiments, the selected therapy can include the combination of any of these therapies.

In some embodiments, the diagnosis of EE can be enhanced by combining information from the quantifying step with one or more other tests for or indicia of EE. The other tests for or indicia of EE can include, for example, determination of allergic status, quantification of biomarkers associated with allergic status, determination of atopic status, quantification of biomarkers associated with atopic status, endoscopy with biopsy analysis, detection of eosinophils, detection of eotaxin-3, detection of eosinophil-derived neurotoxin, detection of IL-5 protein, and the like.

Embodiments of the invention also include a diagnostic kit, test, or array, including materials for quantification of at least two analytes, wherein the at least two analytes can be, for example, any of the cytokines listed in Table 1, any of the cytokines listed in Table 2, or an mRNA corresponding to any member of the group or its receptor, or the like. In some embodiments, the at least two analytes quantified by the diagnostic kit, test, or array can include, for example, any of the cytokines listed in Table 1, or an mRNA corresponding to any member of the group or its receptor, or the like.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts a series of graphs representing mRNA levels of various cytokines in healthy subjects and in patients with EE. Expression levels were quantified using real-time PCR and were normalized to the housekeeping gene glyceraldehyde 3-phosphate dehydrogenase (GAPDH) and expressed as a relative ratio. P values were calculated by using the Mann-Whitney test: *P<0.05; ***P<0.0005.

FIG. 2 depicts a series of graphs representing mRNA levels of eotaxin-3, IL5, IL4, IL13, and IL5RA in healthy subjects (normal [NL]) and in patients with chronic esophagitis (CE) and EE; patients with EE were further subdivided on the basis of the maximum eosinophil number in the biopsy into active (>23 eosinophils/hpf, Active EE), intermediate (1-23 eosinophils/hpf, Int EE), and inactive (0 eosinophils/hpf, Inactive EE). Expression levels were quantified using real-time PCR and were normalized to GAPDH and expressed as a relative ratio. P values were calculated by using the Kruskal-Wallis test with a Dunn multiple comparison test: *P<0.05; **P<0.005; ***P<0.0005.

FIG. 3 depicts a series of graphs representing expression of eotaxin-3, IL5, IL4, IL13, and IL5RA in patients with active EE with and without allergy. Expression levels were quantified using real-time PCR and were normalized to GAPDH and expressed as a relative ratio. P values were calculated by using the Mann-Whitney test: **P<0.005; ***P<0.0005.

FIG. 4 depicts a series of graphs representing correlation and linear regression between eotaxin-3, IL5, IL4, and IL13. Spearman correlation r and P values were calculated to test correlation between the ranked cytokine levels. Linear regression curves are presented with the corresponding P value and r² on the curves to test whether the cytokine levels directly correlate with each other. A nonsignificant P value (NS) indicates that the slope of the curve is not significantly different from 0.

FIG. 5A depicts a series of graphs representing various plasma cytokine levels in healthy subjects and in patients with EE. FIG. 5B depicts a series of graphs representing various plasma cytokine levels in EE patients before and after therapy. The P values were calculated by using the Mann-Whitney test for FIG. 5A and the paired t test for FIG. 5B.

FIGS. 6A and 6B depict a series of graphs representing various plasma cytokine levels in healthy subjects and in patients with EE. The P values>0.05 were calculated by using the Mann-Whitney test.

FIGS. 7A-D represent various plasma cytokine levels (in pg/mL) in healthy subjects and in patients with EE before and after therapy.

### DETAILED DESCRIPTION OF THE INVENTION

All references cited herein are incorporated by reference in their entirety. Also incorporated herein by reference in their entirety include: U.S. Patent Application No. 60/633,909, EOTAXIN-3 IN EOSINOPHILIC ESOPHAGITIS, filed on Dec. 27, 2004; U.S. Pat. No. 8,030,003, DIAGNOSIS OF EOSINOPHILIC ESOPHAGITIS BASED ON PRESENCE OF AN ELEVATED LEVEL OF EOTAXIN-3, issued Oct. 4, 2011 and filed as U.S. patent application Ser. No. 11/721,127 on Jun. 7, 2007; U.S. patent application Ser. No. 12/492,456, EVALUATION OF EOSINOPHILIC ESOPHAGITIS, filed on Jun. 26, 2009; U.S. patent application Ser. No. 12/628,992, IL-13 INDUCED GENE SIGNATURE FOR EOSINOPHILIC ESOPHAGITIS, filed on Dec. 1, 2009; U.S. Patent Application No. 61/436,907, EPIGENETIC REGULATION OF THE IL-13-INDUCED HUMAN EOTAXIN-3 GENE BY CBP-MEDIATED HISTONE 3 ACETYLATION, filed on Jan. 27, 2011; U.S. patent application Ser. No. 13/051,873, METHODS AND COMPOSITIONS FOR MITIGATING EOSINOPHILIC ESOPHAGITIS BY MODULATING LEVELS AND ACTIVITY OF EOTAXIN-3, filed on Mar. 18, 2011; U.S. patent application Ser. No. 13/132,884, DETERMINATION OF EOSINOPHILIC ESOPHAGITIS, filed on Jun. 3, 2011; U.S. Patent Application No. 61/571,115, DIAGNOSTIC METHODS OF EOSINOPHILIC ESOPHAGITIS, filed on Jun. 21, 2011; and, U.S. patent application Ser. No. 13/132,295, METHODS OF DETERMINING EFFICACY OF GLUCOCORTICOID TREATMENT OF EOSINOPHILIC ESOPHAGITIS, filed on Aug. 22, 2011.

Unless otherwise noted, technical and scientific terms are to be understood according to conventional usage by those of ordinary skill in the relevant art to which this invention belongs.

Non-invasive techniques for the diagnosis of EE, such as biomarker detection methods, would be preferable to endoscopic techniques. Such non-invasive techniques are not currently used due to the low sensitivity and specificity of available EE biomarkers.

Therapies for EE include allergen removal, steroid treatment, dietary management, and the combination of steroid treatment and dietary management. Other EE therapies include the use of proton pump inhibitors (PPIs), topical glucocorticoids, such as fluticasone or budesonide, humanized antibodies against relevant cytokines, such as eotaxin-3, IL-13, and IL-5, and small molecule inhibitors of an eosinophil and/or allergic disease activation pathway, such as a prostaglandin D2, IL-4, or IL-13 antagonist.

As disclosed herein, certain cytokines/genes can be associated with EE, and their plasma or serum levels can be measured to provide or contribute to an EE diagnosis.

EE diagnosis typically requires endoscopy with biopsy analysis because reliable, noninvasive biomarkers for EE have not yet been identified. While blood levels of eosinophils, eotaxin-3, eosinophil-derived neurotoxin, and IL-5 proteins are known to be elevated in EE, their sensitivity and specificity are generally too low to be clinically helpful (see, e.g., Konikoff M. et al. Gastroenterology 131:1381-91 (2006)). Although several phenotypic subsets of EE patients have emerged, EE esophageal transcriptome analysis has revealed a highly conserved expression profile irrespective of patient phenotype (as defined by sex, atopic status, and familial clustering), but the sensitivity of the EE transcriptome has not been determined (see, e.g., Blanchard, C. et al. J. Allergy Clin. Immunol. 118:1054-9 (2006); Blanchard, C. and Rothenberg, M. Gastrointest. Endosc. Clin. N. Am. 18:133-43 (2008)).

Early studies in mice have indicated that esophageal eosinophilia occurs in T_{H}2 inflammatory responses (see, e.g., Mishra, A. et al. J. Clin. Invest. 107:83-90 (2001); Mishra, A. et al. J. Immunol. 168:2464-9 (2002); Mishra, A. and Rothenberg, M. Gastroenterology 125:1419-27 (2003)). However, the local and systemic expression of relevant cytokines has not been well characterized, and the expression of T_{H}2 cytokines in patients with EE has been reported in only a few studies (see, e.g., Prussin, C. et al. J. Allergy Clin. Immunol. 124:1326-32 (2009); Straumann, A. et al. J. Allergy Clin. Immunol. 108:954-61 (2001); Schmid-Grendelmeier, P. et al. J. Immunol. 169:1021-7 (2002); Blanchard, C. et al. J. Clin. Invest. 116:536-47 (2006); Blanchard, C. et al. J. Allergy Clin. Immunol. 120:1292-300 (2007); Aceves, S. et al. J. Allergy Clin. Immunol. 119:206-12 (2007); Gupta, S. et al. J. Pediatr. Gastroenterol. Nutr. 42:22-6 (2006); Bullock, J. et al. J. Pediatr. Gastroenterol. Nutr. 45:22-31 (2007)). Characterization of gene expression differences between patients with EE and non-EE subjects via esophageal microarray expression analysis has established eotaxin-3 as the most overexpressed gene in patients with EE; this finding has been replicated in independent studies (see, e.g., Blanchard, C. et al. Int. J. Biochem. Cell Biol. 37:2559-73 (2005); Bhattacharya, B. et al. Hum. Pathol. 38:1744-53 (2007); Lucendo, A. et al. Am. J. Gastroenterol. 103:2184-93 (2008)).

Immunologic cytokines are often produced at levels below the detection capabilities of genome-wide expression chips. For example, although IL13 is not part of the initial EE transcriptome identified by microarray analysis of esophageal tissue (see, e.g., Blanchard, C. et al. J. Clin. Invest. 116:536-47 (2006)), real-time PCR has been used to demonstrate that patients with EE display a 16-fold increase in esophageal IL13 compared with control individuals (see, e.g., Blanchard, C. et al. J. Allergy Clin. Immunol. 120:1292-300 (2007)).

As described herein, the expression of a panel of potentially relevant cytokines in esophageal biopsies from a cohort of patients with EE and healthy subjects was examined. Select genes associated with the cytokines deemed to be potentially relevant to EE were examined in a larger cohort of EE patients and healthy subjects.

The relationship between these cytokines and other biomarkers associated with EE was examined, as well as the impact of clinical parameters on the expression of these genes. These clinical parameters include atopy, allergic status, and eosinophil levels.

Plasma cytokine levels were also examined for their relevance in the diagnosis of EE, and were compared with unaffected controls with and without allergy. Cytokine expression levels were determined in patients with and without EE in the esophageal mucosa and the blood. New cytokines not previously associated with EE, such as IL1F9 and CCL23, have been found to be up-regulated in EE compared with healthy patients.

Although EE diagnosis is complex, only 8.7% of active EE samples had an eotaxin-3 level that overlapped with healthy samples using only a single biopsy sample per patient.

Correlations were found between mRNA levels of the T_{H}2 cytokines IL13, IL5, and eotaxin-3, but IL4 was not found to correlate with IL13 or eotaxin-3 levels. The allergic status was an important confounder because IL4 and IL5 mRNA were increased in patients with allergy and EE. Except for the eosinophil level, none of the clinical parameters analyzed (therapy, allergic status, sex) was able to explain the inter-patient variability of eotaxin-3 and IL13 levels in patients with active EE. The establishment of a scoring panel based on plasma levels, including 8 cytokines, was able to predict diagnosis with 79% positive predictive value, 68% negative predictive value, 83% specificity, and 61% sensitivity in this population of patients referred for endoscopy.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of subjects with a positive test result who are correctly diagnosed; for tests with a high PPV, a positive test indicates the presence of the condition in question. The NPV of a particular diagnostic test represents the proportion of subjects with a negative test result who are correctly diagnosed; for tests with a high NPV, a negative test indicates the absence of the condition. Sensitivity represents the proportion of correctly identified subjects who are actual positives; for tests with high sensitivity, a positive test indicates the presence of the condition in question. Specificity represents the proportion of correctly identified subjects who are actual negatives; for tests with high specificity, a negative test indicates the absence of the condition.

As described herein, cytokine levels of nearly 300 patients were analyzed, and the overlap among cytokine levels was assessed. Real-time PCR was used to demonstrate with 89% sensitivity that eotaxin-3 mRNA expression in patients with EE is increased compared with control patients. Previous histopathologic studies indicate that a minimum of 5 biopsies are required to achieve 100% sensitivity for diagnosis of EE, with a single biopsy only achieving 55% sensitivity (see, e.g., Shah, A. et al. Am. J. Gastroenterol. 104:716-21 (2009); Gonsalves, N. et al. Gastrointest. Endosc. 64:313-9 (2006)). As further described herein, results were obtained by using only a single RNA sample per patient, indicating that molecular diagnosis can be useful for disease diagnosis.

As described herein, cytokine correlations reveal the concerted expression of IL13, IL5, and IL4 mRNA and indicate expression in the same cell type, such as a T_{H}2 cell producing IL-13 and IL-5. IL-13 has been shown to specifically induce eotaxin-3 in esophageal epithelial cells (see, e.g., Blanchard, C. et al. J. Allergy Clin. Immunol. 120:1292-300 (2007)), and a recent study (Prussin, C. et al. J. Allergy Clin. Immunol. 124:1326-32 (2009)) has emphasized the presence of unique food antigen-specific, IL-5-positive T_{H}2 cells in patients with eosinophil-associated gastrointestinal disorders compared with patients with food anaphylaxis. The implications of IL-5 and IL-13 in EE have also been demonstrated in murine EE models (see, e.g., Mishra, A. et al. J. Clin. Invest. 107:83-90 (2001); Mishra, A. et al. J. Immunol. 168:2464-9 (2002); Mishra, A. and Rothenberg, M. Gastroenterology 125:1419-27 (2003)). Although IL5RA mRNA was up-regulated in patients with active EE, its low expression level can explain why it did not correlate with eosinophil levels. IL4 and IL5 are dysregulated in patients with allergy and EE compared with patients without allergy with EE, and these increases can reflect the systemic allergic history of the patients rather than the local activity of the disease as reflected by eotaxin-3 and IL-13 expression levels.

A recent study (Yamazaki et al. Dig. Dis. Sci. 51:1934-41 (2006)) has shown that common food and environmental allergens induce increased production of IL-13 and IL-5 by PBMCs after stimulation with aeroallergens or food allergens in patients with EE compared with healthy individuals. As described herein, in the study of patients referred for endoscopy, the establishment of a plasma scoring panel including 8 cytokines was able to predict diagnosis of EE with 79% positive predictive value, 68% negative predictive value, 83% specificity, and 61% sensitivity.

As described herein, although evidencing relatively high scores, these results also indicate that patients with an allergic history, who are challenging to diagnose, can result in false-positive occurrences. In addition, the positive predictive value is reflective of the study population (potential patients with EE) that was composed of about 50% non-EE and 50% EE in the cohort. In the general population, where the prevalence of EE is lower, the positive predictive value would thus be lowered. Although the cytokine dysregulation was not reproduced in the prospective study, specificity and sensitivity were relatively high because of the high threshold levels chosen, which were set above the maximum level observed in the non-EE group.

The potential roles of the cytokines that were significantly modified in EE compared with healthy subjects is of interest. For example, CXCL14 down-regulation has also been shown in squamous head and neck cancer and has an anti-proliferative role on epithelial cells. The specific epithelial growth factor receptor tyrosine kinase inhibitor, which restores CXCL14 expression in head and neck squamous cell carcinoma (see, e.g., Ozawa, S. et al. Biomed. Res. 30:315-8 (2009); Ozawa, S. et al. Cancer Sci. 100:2202-9 (2009)), can contribute to a decrease in esophageal epithelial cell proliferation in patients with EE. In contrast, CCL23 mRNA is increased in EE and has been shown to be induced after signal transducer and activator of transcription (STAT) 6 activation (see, e.g., Novak, H. et al. J. Immunol. 178:4335-41 (2007)): CCL23 is involved in endothelial cell proliferation, a feature that can contribute to the papillae elongation observed in EE. Dysregulation of novel cytokines and receptors in EE has also been identified. Marked changes in IL-1 family-related molecules have been noted with up-regulation of IL1B and IL-1-related family member 6 and down-regulation of the inhibitory receptor (IL1RA) and IL-1-related family member 9. Thus, EE can involve coordinate pro-inflammatory signals triggered by IL-1-related molecules, indicating the importance of post-IL-1 receptor signaling (such as nuclear factor-κB). The EE transcriptome has evidence for activation of this pathway via overexpression of IL8, monocyte chemotactic protein-2, and TNF-alpha induced protein 6 (see, e.g., Blanchard, C. et al. J. Clin. Invest. 116:536-47 (2006)).

As described herein, the molecular pathogenesis of EE has been explored by identifying esophageal over-expression of a panel of chemokines and cytokines in addition to the previously reported IL13 and eotaxin-3. Although the screening array encoded 84 relevant mRNAs, only approximately 20% were dysregulated in EE. A strong correlation was identified among IL13, IL5, and eotaxin-3 but not IL4 mRNA levels, consistent with the presence of an IL-13-producing T_{H}2 cell population. Using molecular analysis of only eotaxin-3 in a large cohort of patients, approximately 90% sensitivity for diagnosis was obtained. Furthermore, blood levels of the core panel of 8 cytokines reached moderate specificity and sensitivity regardless of the global increase of these cytokines in the different groups of patients. However, atopy was a confounder for systemic cytokine levels. IL13 and IL5 associate with eosinophil and eotaxin-3 levels, indicating the key role of adaptive T_{H}2 immunity in regulating eotaxin-3-driven esophageal eosinophilia in the absence of a consistent systemic change in cytokines.

The clinical value includes the finding that the pathogenesis of EE involves a dysregulated local cytokine network in the esophageal mucosa and elevated eotaxin-3 expression (89% sensitivity in a single biopsy) in the absence of consistent systemic changes in cytokines.

Certain embodiments of the invention include using quantification data from a gene-expression analysis and/or from a cytokine analysis, either from an esophageal biopsy sample or from a sample of esophageal mucosa or from a blood sample. Embodiments of the invention include not only methods of conducting and interpreting such tests but also include reagents, kits, assays, and the like, for conducting the tests.

The correlations disclosed herein, between EE and cytokine levels and/or mRNA levels, provide a basis for conducting a diagnosis of EE, or for enhancing the reliability of a diagnosis of EE by combining the results of a quantification of cytokine or mRNA with results from other tests or indicia of EE. Thus, even in situations in which a given cytokine or mRNA correlates only moderately or weakly with EE, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the invention are expressly contemplated to be used both alone and in combination with other tests and indicia, whether quantitative or qualitative in nature.

The disclosure, figures, and tables herein make mention of statistical significance and “p values.” While p values below 0.05 are considered to be statistically significant, it is within the scope of embodiments of the present invention to make use of correlations having a reported p value above 0.05 as well as below 0.05. For example, in a study having a small sample size but a genuine correlation, a p value can be above 0.05, such as, for example, 0.06, 0.07, 0.08, 0.09, 0.10, 0.15, or more. Since p value is affected by sample size, two studies can have the same proportion of outcomes, and a study with a smaller sample size can have a p value above 0.05, while the study with the larger sample size can have a p value below 0.05, even though the correlation is proportionally the same. Thus, while a p value below 0.05, for any sample size, is a strong indication of a statistically significant correlation, a genuine correlation can exist, that is tested with a small sample size, and the p value of such a test can be above 0.05.

The illustrative embodiments described in the detailed description, drawings, and claims are not meant to be limiting. Having described the invention in detail, it will be apparent that modifications, variations, and equivalent embodiments are possible without departing from the spirit or scope of the subject matter presented herein.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Comparison of Blood Cytokine Levels Between Healthy Subjects and EE Patients

### Comparison of Healthy Subjects and EE Patients

A study was undertaken on patients referred for endoscopy to determine the levels of various cytokines in their serum. Patients with no histologic findings in the gastrointestinal tract and who presented with a healthy esophagus with no histological abnormality were defined as healthy.

Patients were classified into discovery and replication cohorts and were studied to determine their expression levels of relevant RNA. The discovery cohort was composed of 5 healthy subjects and 5 untreated patients with EE. The replication cohort was composed of 11 healthy subjects and 11 patients with EE who had not received steroid treatment.

Patients diagnosed with GERD or CE were regrouped in the CE group. A proportion (47%) of the 226 patients with EE was treated with a proton pump inhibitor (PPI) at the time of the endoscopy. Of the patients who did not receive PPI treatment at the time of the endoscopy (n=120), the patients either did not respond to a treatment including PPI (13%), or the patients did respond to steroids alone (11%), diet management alone (39%), or the combination of the steroids and diet management (33%) in a later endoscopy. No information was available for 5 patients.

Plasma from the blood of those without EE (including healthy subjects and patients with GERD or CE) and patients with EE was used to quantify cytokines in three cohorts: (1) a learning set (n=25) composed of 12 healthy subjects and 13 patients with EE; (2) a before-and-after treatment set (n=5) composed of patients with EE; and (3) a prospectively recruited blind set of patients referred for endoscopy composed of patients without EE and with active EE and excluding treated and partially treated patients with EE (n=36). For research purposes, active EE was defined as patients having >24 eosinophils/hpf in at least 1 hpf.

Blood samples were collected in heparinized tubes and centrifuged (3000 rpm) for 10 minutes at 4° C.; plasma was stored at −70° C. until further use. The allergic status was defined as having present or past history of allergic diseases and/or at least 1 positive skin prick test. Biopsy and blood samples were collected during routine endoscopy or blood draw after informed consent as approved by the institutional review board.

### RNA Extraction and Real-Time PCR Analysis

Total RNA from biopsy samples were stored in RNALater (Qiagen, Valencia, Calif.), then were extracted by using the Qiagen mini RNA extraction kit (Qiagen), and reverse transcription was performed by using Iscript (Bio-Rad, Hercules, Calif.). The reactions for each set of samples were done at different times and produced different yields, leading to variations in the detection limits of the different data sets. Real-time PCR was performed by rapid cycling using the ready-to-use IQ5 SYBR mix (Bio-Rad) according to the manufacturer's instructions. PCR products were sequenced at the Cincinnati Children's Hospital Medical Center sequencing core facility.

The PAHS-011 Human Inflammatory Cytokine and Receptor Array (SABiosciences, Frederick, Md.) was used in 5 healthy subjects and 5 patients with EE by interrogating the following: chemokine genes (component of complement 5 (C5), CCL1 [1-309], CCL11 [eotaxin], CCL13 [macrophage chemoattractant protein (MCP-4)], CCL15 [macrophage inflammatory protein (MIP-1d)], CCL16 [human CC chemokine (HCC-4)], CCL17 [TARC], CCL18 [pulmonary and activation-regulated chemokine (PARC)], CCL19, CCL2 [MCP-1], CCL20 [MIP-3a], CCL21 [MIP-2], CCL23 [myeloid progenitor inhibitory factor 1 (MPIF-1)], CCL24 [MPIF-2/eotaxin-2], CCL25 [thymus-expressed chemokine TECK)], CCL26 [eotaxin-3], CCL3 [MIP-1 a], CCL4 [MIP-1β], CCL5 [regulated on activation normal T cell expressed and secreted (RANTES)], CCL7 [MCP-3], CCL8 [MCP-2], CXCL1, CXCL10 [IP-10], CXCL11 [interferon-inducible T cell (I-TAC)/interferon gamma-induced protein 10 kDa (IP-9)], CXCL12 [stromal cell-derived factor-1 (SDF1)], CXCL13, CXCL14, CXCL2, CXCL3, CXCL5 [epithelial neutrophil-activating protein ENA-78)/LPS-induced CXC chemokine (LIX)], CXCL6 [granulocyte chemotactic protein-2 GCP-2)], CXCL9, and IL8), chemokine receptor genes (CCL13 [MCP-4], CCR1, CCR2, CCR3, CCR4, CCR5, CCR6, CCR7, CCR8, CCR9, CX3CR1, IL8RA, and XCR1 [CCXCR1]), cytokine genes (CD40Ligand [TNF ligand superfamily member 5 (TNFSF5)], IFNA2, IL10, IL13, IL17C, IL1A, IL1B, IL1F10, IL1F5, IL1F6, IL1F7, IL1F8, IL1F9, IL22, IL5, IL9, LTA, LTB, MIF, small cytokine E1 (SCYE1), secreted phosphoprotein 1 (SPP1), and TNF), cytokine receptor genes (IL10RA, IL10RB, IL13RA, IL13RA1, IL5RA, and IL9R), and other genes involved in inflammatory responses (ABCF1, BCL6, C3, C4A, CCAAT/enhancer-binding protein beta (CEBPB), C-reactive protein (CRP), ICEBERG, IL1R1, IL1RN, IL8RB, leukotriene B4 Receptor (LTB4R), and Toll-interacting protein TOLLIP)). Results were analyzed by using the web-based software found at http <colon slash slash> www <dot> sabiosciences <dot> com <slash> per <slash> arrayanalysis <dot> php.

### Multiplex Analysis for Quantification of Blood Cytokine Levels

The 29-plex Lincoplex human cytokine kit (Millipore, Billerica, Mass.) was used to quantify serum levels of the following cytokines IL-1β, IL-2, IL-1Rα, IL-4, IL-5, EGF, IL-6, IL-7, IL-8, IL-10, TGF-α, fractalkine, IL-12p70, IL-13, IL-15, IL-17, IL-1α, IFN-γ, granulocyte colony-stimulating factor (G-CSF), granulocyte-macrophage colony-stimulating factor (GM-CSF), TNF-α, eotaxin-1, MCP-1, CD40L, IL-12p40, MIP-1α, MIP-1β, IP-10, and VEGF. Samples were run in duplicate for the learning set and the before-and-after treatment set.

Lower and upper detection limits were 3.2 pg/mL and 10,000 pg/mL, respectively. Data with levels lower than 3.2 pg/mL were adjusted to 3.2 pg/mL, and data with values higher than 10,000 pg/mL were adjusted to 10,000 pg/mL.

For the prospectively recruited blind set of patients, patients were collected prospectively, and the investigator was unblinded only at the end of the analysis. These samples were subjected to the 39-plex Milliplex human cytokine panel (Millipore), including fibroblast growth factor (FGF-2), FMS-like tyrosine kinase 3 receptor ligand (FLT-3L), GRO, IFN-α2, IL-3, IL-9, MCP-3, macrophage-derived chemokine (MDC), sIL-2Rα, and TNF-β in addition to the 29-plex. Samples run in the first analysis were incorporated in the second quantification to check for reproducibility. All cytokines tested were no more than 18% different between the two runs except for CD40L, which was decreased by 45% in the third set.

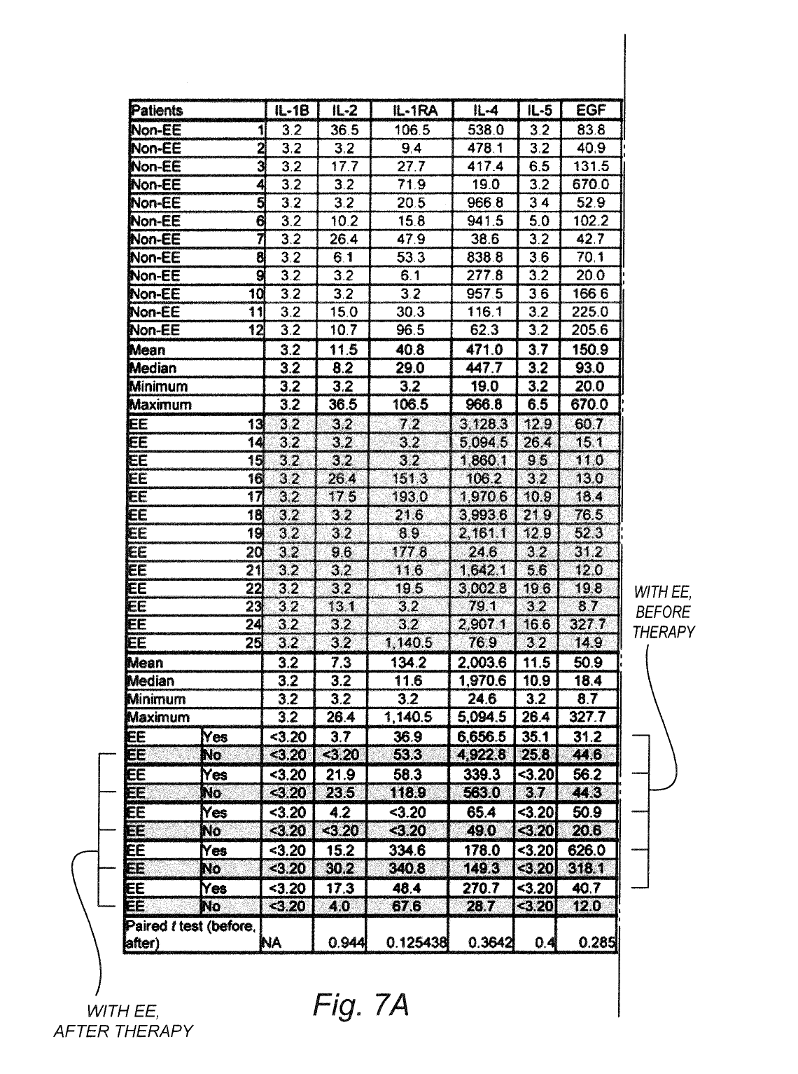

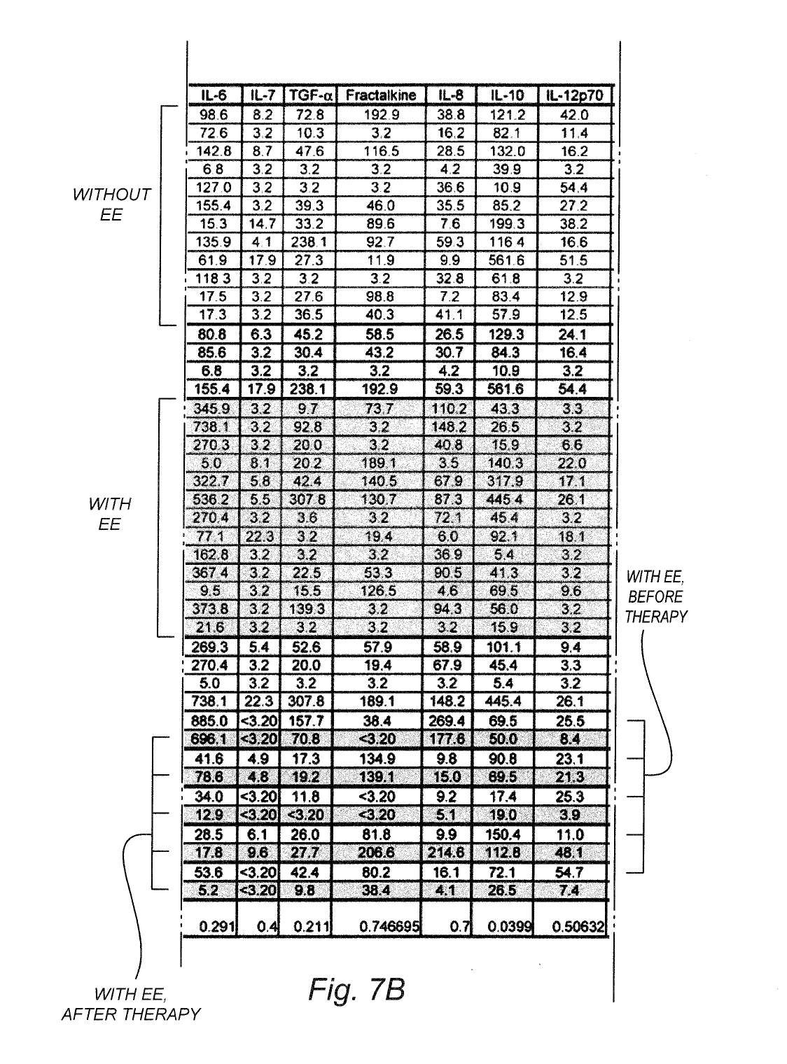

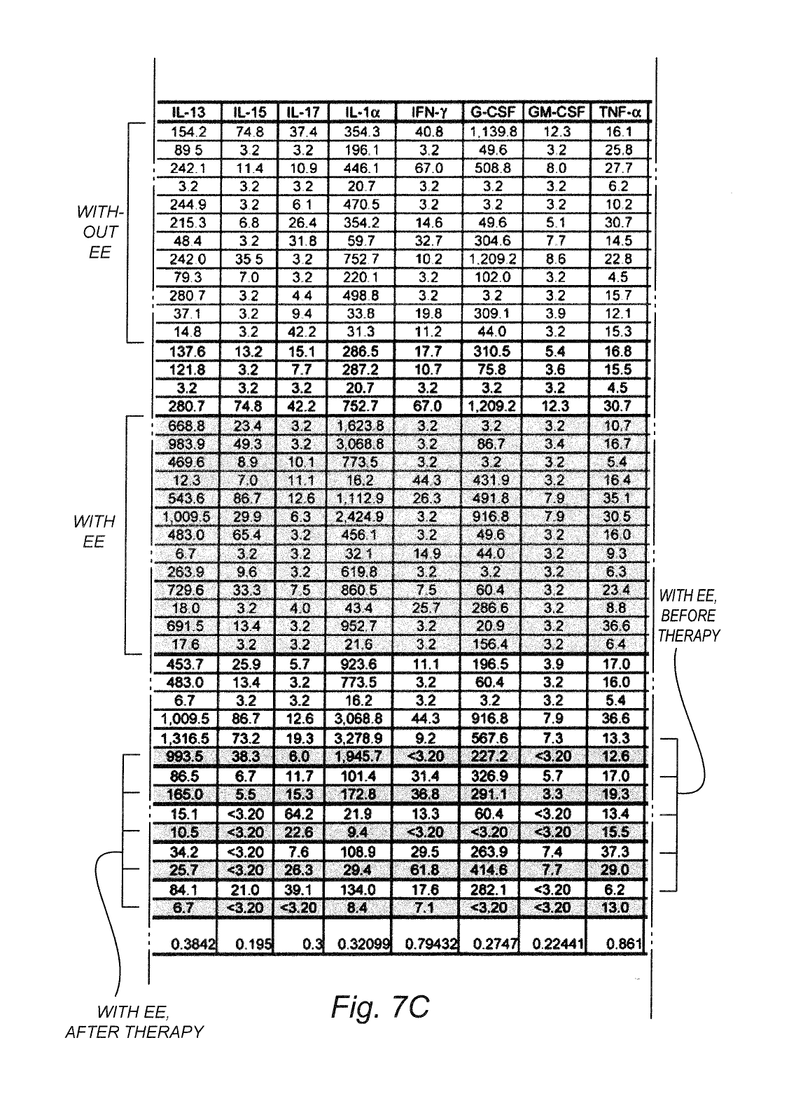

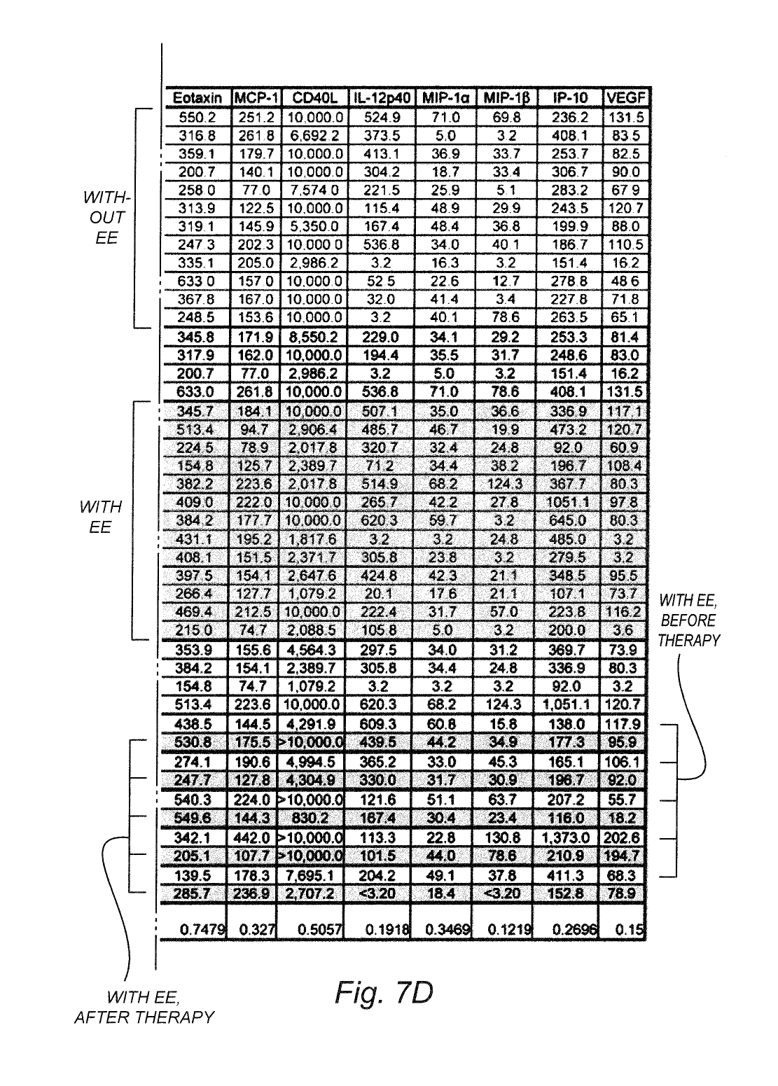

A scoring system based on a panel of cytokines was established, adding 1 to a patient's score for each up-regulation or down-regulation of specific cytokines Cytokine up-regulation was indicated for cytokine values higher than the maximum value observed in the healthy subjects for the following cytokine levels, measured in pg/mL: IL-1a>753; IL-4>967; IL-5>7; IL-6>155; IL-13>281. Cytokine down-regulation was indicated for cytokine values lower than the minimum value observed in healthy subjects for the following cytokine level, measured in pg/mL: CD40L<2986. Cytokine down-regulation was also indicated for cytokine values lower than the average observed in healthy subjects when at least 1 healthy subject was below the detection limit for the following cytokine levels, measured in pg/mL: IL-12p70<24; IL-17<15; see FIGS. 7A-D. Patients with a score of 3 or more were classified as having EE with 100% sensitivity and 100% specificity; see Table 1.

TABLE 1
Establishment of the retrospective scoring panel

| | | (heterodimer) | | | | | | | | |
|---|---|---|---|---|---|---|---|---|---|---|
| | | SEQ ID | SEQ ID | SEQ ID | SEQ ID | SEQ ID | SEQ ID | SEQ ID | SEQ ID | |
| Threshold | | NO 1 | NO 2 | NO 3 | NOs 4 and 5 | NO 6 | NO 7 | NO 8 | NO 9 | |
| cytokine | | IL-4 | IL-5 | IL-6 | IL-12p70 | IL-13 | IL-17 | IL-1α | CD40L | |
| levels | Patient | >966.76 | >6.53 | >155.38 | <24.09 | >280.72 | <15.12 | >752.71 | <2986.16 | Score |
| Non-EE | 1 | 0 | 0 | 0 | 0 | 0 | 0 | 0 | 0 | 0 |
| Non-EE | 2 | 0 | 0 | 0 | 1 | 0 | 1 | 0 | 0 | 2 |
| Non-EE | 3 | 0 | 0 | 0 | 1 | 0 | 1 | 0 | 0 | 2 |
| Non-EE | 4 | 0 | 0 | 0 | 1 | 0 | 1 | 0 | 0 | 2 |
| Non-EE | 5 | 0 | 0 | 0 | 0 | 0 | 1 | 0 | 0 | 1 |
| Non-EE | 6 | 0 | 0 | 0 | 0 | 0 | 0 | 0 | 0 | 0 |
| Non-EE | 7 | 0 | 0 | 0 | 0 | 0 | 0 | 0 | 0 | 0 |
| Non-EE | 8 | 0 | 0 | 0 | 1 | 0 | 1 | 0 | 0 | 2 |
| Non-EE | 9 | 0 | 0 | 0 | 0 | 0 | 1 | 0 | 0 | 1 |
| Non-EE | 10 | 0 | 0 | 0 | 1 | 0 | 1 | 0 | 0 | 2 |
| Non-EE | 11 | 0 | 0 | 0 | 1 | 0 | 1 | 0 | 0 | 2 |
| Non-EE | 12 | 0 | 0 | 0 | 1 | 0 | 0 | 0 | 0 | 1 |
| EE | 13 | 1 | 1 | 1 | 1 | 1 | 1 | 1 | 0 | 7 |
| EE | 14 | 1 | 1 | 1 | 1 | 1 | 1 | 1 | 1 | 8 |
| EE | 15 | 1 | 1 | 1 | 1 | 1 | 1 | 1 | 1 | 8 |
| EE | 16 | 0 | 0 | 0 | 1 | 0 | 1 | 0 | 1 | 3 |
| EE | 17 | 1 | 1 | 1 | 1 | 1 | 1 | 1 | 1 | 8 |
| EE | 18 | 1 | 1 | 1 | 0 | 1 | 1 | 1 | 0 | 6 |
| EE | 19 | 1 | 1 | 1 | 1 | 1 | 1 | 0 | 0 | 6 |
| EE | 20 | 0 | 0 | 0 | 1 | 0 | 1 | 0 | 1 | 3 |
| EE | 21 | 1 | 0 | 1 | 1 | 0 | 1 | 0 | 1 | 5 |
| EE | 22 | 1 | 1 | 1 | 1 | 1 | 1 | 1 | 1 | 8 |
| EE | 23 | 0 | 0 | 0 | 1 | 0 | 1 | 0 | 1 | 3 |
| EE | 24 | 1 | 1 | 1 | 1 | 1 | 1 | 1 | 0 | 7 |
| EE | 25 | 0 | 0 | 0 | 1 | 0 | 1 | 0 | 1 | 3 |

### Statistical Analysis

Statistical analysis was performed on the results, with data expressed as mean+/−SD. Statistical significance comparing different treatments or groups was determined by the Student t test (normal distribution, equal variance), the Welch t test (normal distribution, unequal variances), the Mann-Whitney test (non-parametric test, 2 groups), the Kruskal-Wallis test followed by a Dunn multiple comparison test (non-parametric test, 3 groups or more), or a paired t test (for quantification of cytokines before and after therapy in the same patients) using Prism 4 GraphPad Software (Palo Alto, Calif.). Non-parametric (ranked) correlations were calculated using Spearman correlations. Linear regressions were then calculated, and P values were assessed to test the hypothesis that a linear correlation exists with a slope different from 0.

### Example 2

### Expression Of Cytokine and Cytokine-Receptor mRNA in Esophageal Biopsies from Healthy Subjects and Patients with EE

In the same study, the Human Inflammatory Cytokine & Receptor PCR Array (SABiosciences) was used to quantify the expression levels of 84 key genes involved in the inflammatory response in esophageal biopsies from a discovery cohort with 5 representative patients with EE and 5 representative healthy control subjects (Table 2). Of the 84 genes present on the array, the expression of 21 genes was modified by more than 4-fold in EE compared with healthy patient biopsies; of these 21 genes, 19 genes were up-regulated, and 2 genes were down-regulated. One gene was significantly down-regulated but not modified by more than 4-fold (Table 2). The up-regulated genes included eotaxin-3 (69-fold expression increase); ATP-binding cassette, subfamily F, member 1 (18-fold); chemokine (C-X-C motif) ligand 1 (growth-regulated protein alpha [GROA]; 16-fold); chemokine (C-C motif) ligand 23 (macrophage inflammatory protein 3) and IL1B (7-fold each); IL1F9 (6-fold); CD40L, CXCL2, CCR5, and CXCL3 (5-fold each); and IL5RA, CCL1, CCL20, BCL6, and IL17C (4-fold each). The down-regulated genes were chemokine (C-X-C motif) ligand 14 (breast and kidney-expressed chemokine [BRAK]; 9-fold); IL-1 family, member 6 (IL1F6; 4-fold); and IL-1 receptor antagonist (IL1RN; 2-fold). While eotaxin-3, IL8, CXCL1, and IL1B have been found to be up-regulated in previous studies by using microarray analysis, the present study has demonstrated dysregulation of several other genes that were not previously suspected (Table 2).

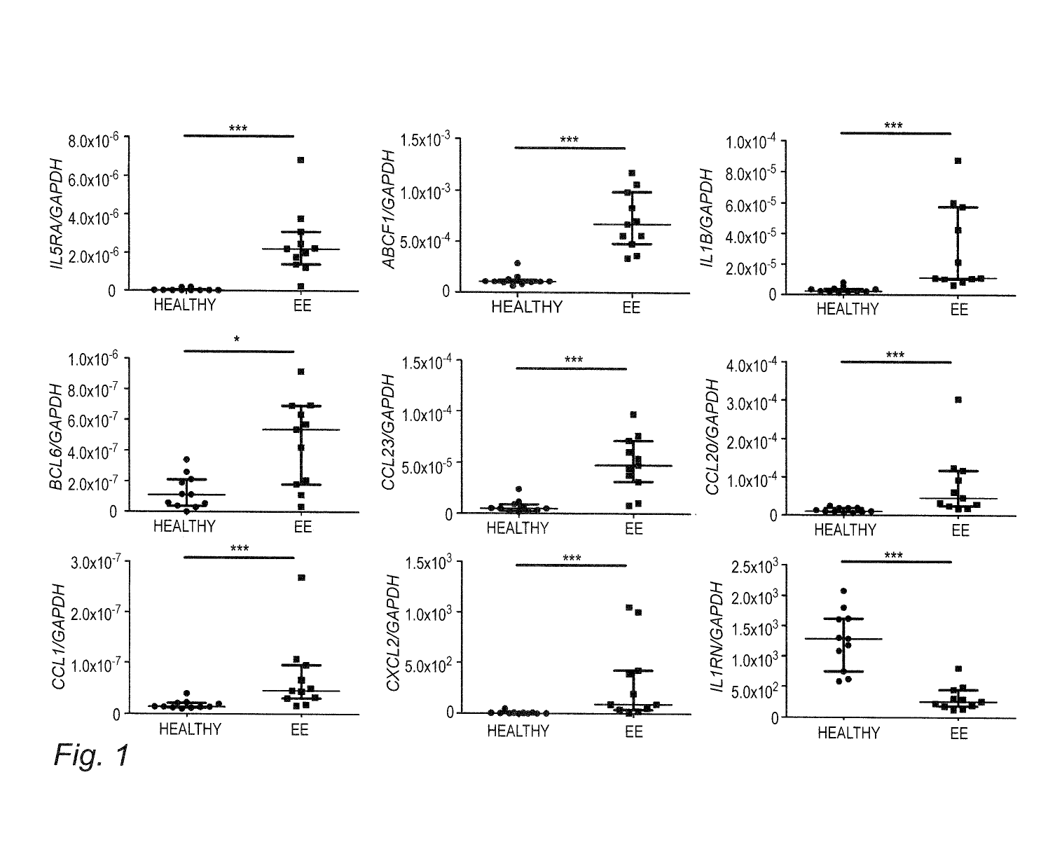

Although increased by more than 4-fold, few genes reached significance, likely due to the sample size (healthy subjects, n=5; EE, n=5). Most gene expression levels were confirmed by real-time PCR and reached significance in a replication cohort with a larger sample size (healthy subjects, n=11; EE, n=11). In the replication cohort, the differential expression of most of the genes identified in the discovery cohort was substantiated, including IL1B, IL1RN, IL5RA, and CCL1 (Table 2; FIG. 1).

TABLE 2
Cytokines with a greater than 4-fold change in expression in EE compared with control group or with a P value <.05

| | | Discovery cohort | | Replication cohort | | |
|---|---|---|---|---|---|---|
| | | PCR array* | | real-time PCR† | | |
| SEQ ID NO | Symbol | Fold change | P value | Fold change | P value | Description |
| 10 | CCL26 | 70 | .0012 | 67 | <.0001 | Chemokine (C-C motif) ligand 26 (eotaxin-3) |
| 11 | ABCF1 | 18 | .30 | 5.8 | <.0001 | ATP-binding cassette, subfamily F, member 1 |
| 12 | CXCL1 | 16 | .018 | 16 | .001 | Chemokine (C—X—C motif) ligand 1 (GRO-α) |
| 13 | CCL23 | 7.3 | .19 | 7.2 | .0003 | Chemokine (C-C motif) ligand 23 (MIP-3) |
| 14 | IL1B | 7.3 | .07 | 5.5 | .0001 | IL-1β |
| 15 | IL8 | 6.8 | .10 | 5.8 | .008 | IL-8 |
| 16 | CCL8 | 6.5 | .24 | 5.4 | .052 | Chemokine (C-C motif) ligand 8 (MCP-2) |
| 17 | IL1F9 | 6.3 | .086 | 3.1‡ | .38 | IL-1 family, member 9 |
| 18 | CD40LG | 5.8 | .23 | ND§ | ND§ | CD40 ligand |
| 19 | CXCL2 | 5.6 | .24 | 19 | .0002 | Chemokine (C—X—C motif) ligand 2 (GRO-β, MIP-2) |
| 20 | CXCL3 | 5.5 | .34 | ND§ | ND§ | Chemokine (C—X—C motif) ligand 3 (GRO-γ, MIP-2b) |
| 21 | CCR5 | 5.2 | .40 | ND§ | ND§ | Chemokine (C-C motif) receptor 5 |
| 22 | IL5RA | 4.9 | .36 | >49¶ | .0001 | IL-5 receptor, α |
| 23 | CCL1 | 4.8 | .36 | 5.3 | .001 | Chemokine (C-C motif) ligand 1 (TCA3) |
| 24 | IL9 | 4.8 | .38 | 5.6‡ | .76 | IL-9 |
| 25 | IL17C | 4.7 | .24 | ND‡§ | ND§ | IL-17C (CX2) |
| 26 | BCL6 | 4.5 | .17 | 3.6 | .013 | B-cell CLL/lymphoma 6 |
| 27 | IL13 | 4.2 | .36 | 16 | <.0001 | IL-13 |
| 28 | CCL20 | 4.0 | .25 | 6.0 | .043 | Chemokine (C-C motif) ligand 20 (MIP-3a) |
| 29 | IL1RN | −2.7 | .011 | −4.0 | .0002 | IL-1 receptor antagonist (IL-1Ra) |
| 30 | IL1F6 | −4.5 | .067 | −9.9 | .0001 | IL-1 family, member 6 (IL-1ε) |
| 31 | CXCL14 | −9.3 | .063 | −5.7 | .003 | Chemokine (C—X—C motif) ligand 14 (BRAK) |

*Discovery cohort, n = 5 healthy and n = 5 EE.
†Replication cohort, n = 11 healthy and n = 11 EE.
‡Levels were undetectable in several patients in both groups.
§Not done or not reproducible.
¶Levels were not detectable in several healthy patients.

### Example 3

### Cytokine And Cytokine Receptor mRNA Expression in Esophageal Biopsies from Healthy Subjects and Patients with EE as a Function of the Activity of the Disease

The mRNA levels of the most up-regulated cytokine (IL13), chemokine (eotaxin-3), and receptor (IL5RA and its ligand IL5) were tested to determine their variability with the degree of activity and within patient groups by using real-time PCR on a large cohort of patients (n=288). The large cohort was composed of healthy subjects and patients who collectively had 288 biopsies collected over 3 years (EE, n=226; healthy, n=14; GERD or CE, n=14, with mean, 6.4, median, 4.5, range, 1-16 eosinophils/hpf; missing or other diagnosis, n=34, were not included in the study). Patients with EE were classified on the basis of their number of eosinophils per hpf (in at least 1 hpf), when available, into active (>24 eosinophils/hpf, n=97), intermediate (1-23 eosinophils/hpf, n=49), or inactive (0 eosinophils, n=52) EE. Patients who had received steroid treatment and/or dietary management were included in these groups.

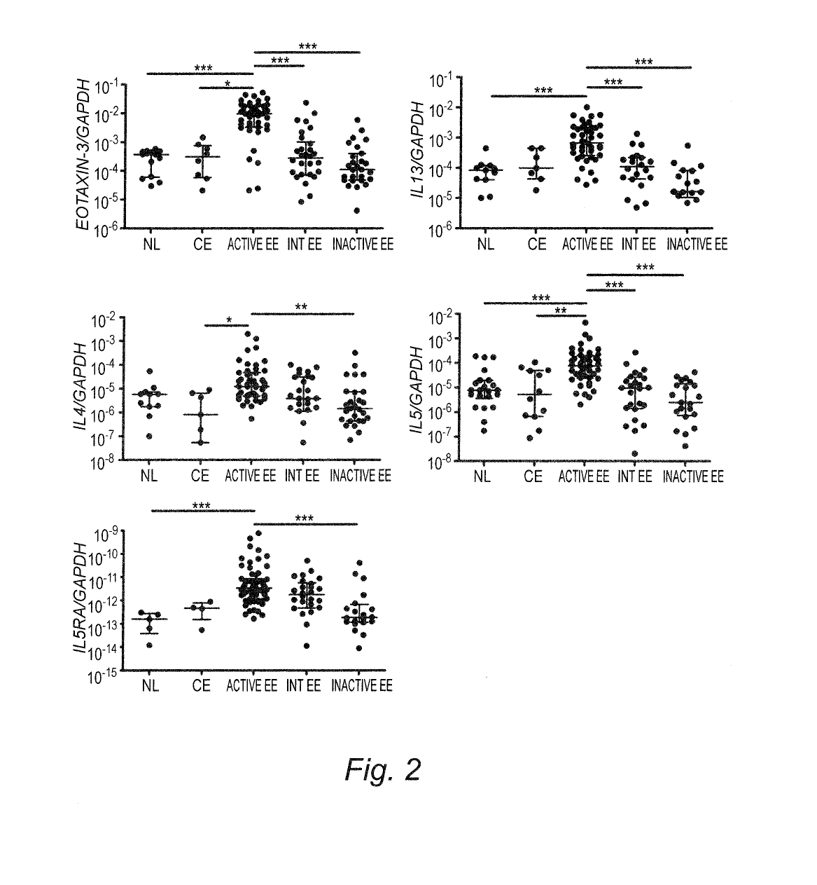

Eotaxin-3 mRNA was the most robust gene overexpressed in patients with active EE (median, 9.7×10⁻³; 25-75 interquartile, 3.3×10⁻³−1.7×10⁻²; see Tables 3A-C) compared with healthy controls (median, 3.7×10⁻⁴; 25-75 interquartile, 6.1×10⁻⁵−4.6×10⁻⁴; P<0.005). In this population, only 5 patients with EE had an eotaxin-3 expression level that overlapped with healthy levels, indicating 89% sensitivity. The activity of the disease was an important factor because patients with partially treated (intermediate) EE, with an intermediate level of eosinophils (1-23 eosinophils/hpf; median, 2.8×10⁻⁴; 25-75 interquartile, 7.2×10⁻⁵−1.0×10⁻³), and patients with successfully treated (inactive) EE, with no esophageal eosinophils (0 eosinophils/hpf; median, 1.1×10⁻⁴; 25-75 interquartile, 4.8×10⁻⁵−4.1×10⁻⁴), did not have significant eotaxin-3 level increases compared with the healthy group. IL13 was significantly up-regulated in active EE compared with healthy subjects (median, 6.7×10⁻⁴; 25-75 interquartile, 2.5×10⁻⁴−2.2×10⁻³ vs median, 8.3×10⁻⁵; 25-75 interquartile, 4.0×10⁻⁵−1.2×10⁻⁴, with 19.5% overlap). Similar to eotaxin-3, IL13 levels in intermediate (median, 1.1×10⁻⁴; 25-75 interquartile, 4.3×10⁻⁵−2.2×10⁻⁴) and inactive EE (median, 1.6×10⁻⁵; 25-75 interquartile, 1.0×10⁻⁵−8.2×10⁻⁵) were not significantly different from healthy levels (FIG. 2). The IL5RA mRNA expression level was significantly up-regulated in patients with active EE compared with healthy controls or patients with inactive EE (FIG. 2). Notably, IL5RA mRNA expression was not detectable in 64% of healthy patients tested, 50% of patients with inactive EE, 33% of patients with intermediate EE, and 22% of patients with active EE. The expression of its ligand, IL5, followed the same trend: IL5 mRNA was significantly increased in patients with active EE versus healthy patients and was lower in patients with intermediate and inactive EE compared with patients with active EE (FIG. 2).

As a control, IL4 mRNA expression showed no significant differences in patients with active EE compared with healthy subjects overall. However, IL4 mRNA levels were significantly decreased by therapies such as glucocorticoids or allergen removal (FIG. 2). In addition, IL2 mRNA was not modified in patients with active EE compared with healthy patients. No significant correlation between eotaxin-3 expression and eosinophil number was observed in the partially treated EE patient group. Only 4 patients with intermediate EE, with 5-15 eosinophils/hpf, had eotaxin-3 expression levels that reached the lower interquartile of eotaxin-3 expression in patients with active EE. No significant difference was observed between the healthy and CE groups for IL13, eotaxin-3, IL5RA, IL5, and IL-4 (FIG. 2), and sensitivity to distinguish CE from EE was similar to that of healthy subjects from EE patients (89%).

TABLE 3A
Cytokine mRNA levels

| | Healthy | Active EE | Intermediate EE | Inactive EE | Nonallergic | Allergic | CE |
|---|---|---|---|---|---|---|---|
| Eotaxin-3 EE | | | | | | | |
| Minimum | 3.0E−05 | 0.0E+00 | 0.0E+00 | 4.2E−06 | 3.3E−04 | 2.5E−04 | 2.1E−05 |
| 25% Percentile | 6.1E−05 | 3.3E−03 | 7.2E−05 | 4.8E−05 | 3.0E−03 | 8.3E−03 | 5.9E−05 |
| Median | 3.7E−04 | 9.8E−03 | 2.8E−04 | 1.1E−04 | 5.8E−03 | 1.3E−02 | 3.1E−04 |
| 75% Percentile | 4.6E−04 | 1.8E−02 | 1.0E−03 | 4.1E−04 | 1.4E−02 | 2.3E−02 | 7.7E−04 |
| Maximum | 5.9E−04 | 5.2E−02 | 2.3E−02 | 5.9E−03 | 2.0E−02 | 4.4E−02 | 1.4E−03 |
| Mean | 3.1E−04 | 1.3E−02 | 1.9E−03 | 5.6E−04 | 8.3E−03 | 1.6E−02 | 4.5E−04 |
| SD | 1.9E−04 | 1.2E−02 | 4.6E−03 | 1.2E−03 | 6.8E−03 | 1.2E−02 | 4.9E−04 |
| SE | 5.2E−05 | 1.8E−03 | 8.5E−04 | 2.3E−04 | 2.2E−03 | 2.3E−03 | 1.7E−04 |
| Lower 95% CI of mean | 2.0E−04 | 9.0E−03 | 1.5E−04 | 8.2E−05 | 3.4E−03 | 1.1E−02 | 4.3E−05 |
| Upper 95% CI of mean | 4.2E−04 | 1.6E−02 | 3.6E−03 | 1.0E−03 | 1.3E−02 | 2.1E−02 | 8.6E−04 |
| Sum | 4.3E−03 | 5.9E−01 | 5.6E−02 | 1.5E−02 | 8.3E−02 | 4.2E−01 | 3.6E−03 |
| IL4 | | | | | | | |
| Minimum | 1.0E−07 | 0.0E+00 | 0.0E+00 | 7.0E−08 | 5.5E−07 | 2.0E−06 | 7.4E−09 |
| 25% Percentile | 1.8E−06 | 4.9E−06 | 1.1E−06 | 4.4E−07 | 3.7E−06 | 6.0E−06 | 5.4E−08 |
| Median | 5.8E−06 | 1.2E−05 | 3.9E−06 | 1.5E−06 | 4.8E−06 | 2.2E−05 | 8.2E−07 |
| 75% Percentile | 7.3E−06 | 4.6E−05 | 3.0E−05 | 7.3E−06 | 6.0E−06 | 9.8E−05 | 6.4E−06 |
| Maximum | 5.4E−05 | 2.0E−03 | 1.0E−04 | 3.1E−04 | 4.5E−05 | 2.0E−03 | 9.0E−06 |
| Mean | 8.8E−06 | 1.1E−04 | 1.8E−05 | 2.1E−05 | 8.8E−06 | 1.8E−04 | 3.0E−06 |
| SD | 1.5E−05 | 3.4E−04 | 2.7E−05 | 6.2E−05 | 1.2E−05 | 4.4E−04 | 3.6E−06 |
| SE | 4.7E−06 | 4.9E−05 | 5.3E−06 | 1.2E−05 | 3.6E−06 | 8.2E−05 | 1.4E−06 |
| Lower 95% CI of mean | −1.5E−06 | 1.1E−05 | 7.6E−06 | −3.5E−06 | 8.7E−07 | 8.3E−06 | −4.0E−07 |
| Upper 95% CI of mean | 1.9E−05 | 2.1E−04 | 2.9E−05 | 4.6E−05 | 1.7E−05 | 3.5E−04 | 6.3E−06 |
| Sum | 9.7E−05 | 5.3E−03 | 5.0E−04 | 5.7E−04 | 1.1E−04 | 5.0E−03 | 2.1E−05 |

TABLE 3B
Cytokine mRNA levels

| | Healthy | Active EE | Intermediate EE | Inactive EE | Nonallergic | Allergic | CE |
|---|---|---|---|---|---|---|---|
| IL5 | | | | | | | |
| Minimum | 1.7E−07 | 2.1E−06 | 2.0E−08 | 4.2E−08 | 2.9E−06 | 0.0E+00 | 8.8E−08 |
| 25% Percentile | 3.7E−06 | 2.4E−05 | 1.4E−06 | 7.5E−07 | 8.4E−06 | 2.9E−05 | 6.7E−07 |
| Median | 7.7E−06 | 7.6E−05 | 9.7E−06 | 2.5E−06 | 3.3E−05 | 1.2E−04 | 5.2E−06 |
| 75% Percentile | 2.0E−05 | 2.0E−04 | 2.6E−05 | 1.5E−05 | 8.7E−05 | 3.1E−04 | 5.0E−05 |
| Maximum | 1.9E−04 | 4.4E−03 | 2.7E−04 | 4.2E−05 | 1.2E−04 | 4.4E−03 | 1.1E−04 |
| Mean | 3.2E−05 | 2.5E−04 | 2.6E−05 | 8.9E−06 | 4.6E−05 | 3.0E−04 | 2.6E−05 |
| SD | 5.9E−05 | 6.8E−04 | 5.5E−05 | 1.2E−05 | 4.2E−05 | 7.0E−04 | 3.5E−05 |
| SE | 1.3E−05 | 1.0E−04 | 1.1E−05 | 2.5E−06 | 1.2E−05 | 1.1E−04 | 1.0E−05 |
| Lower 95% CI of mean | 5.9E−06 | 5.1E−05 | 3.1E−06 | 3.7E−06 | 2.0E−05 | 8.6E−05 | 4.2E−06 |
| Upper 95% CI of mean | 5.8E−05 | 4.6E−04 | 4.8E−05 | 1.4E−05 | 7.1E−05 | 5.2E−04 | 4.9E−05 |
| Sum | 7.1E−04 | 1.2E−02 | 6.4E−04 | 1.9E−04 | 6.0E−04 | 1.3E−02 | 3.2E−04 |
| IL13 | | | | | | | |
| Minimum | 1.0E−05 | 2.8E−05 | 4.9E−06 | 0.0E100 | 8.4E−05 | 0.0E+00 | 1.8E−05 |
| 25% Percentile | 4.0E−05 | 2.5E−04 | 4.3E−05 | 1.0E−05 | 2.4E−04 | 2.5E−04 | 4.3E−05 |
| Median | 8.3E−05 | 6.7E−04 | 1.1E−04 | 1.6E−05 | 5.1E−04 | 6.7E−04 | 9.8E−05 |
| 75% Percentile | 1.2E−04 | 2.2E−03 | 2.2E−04 | 8.2E−05 | 1.2E−03 | 2.2E−03 | 4.4E−04 |
| Maximum | 4.4E−04 | 1.0E−02 | 1.3E−03 | 5.5E−04 | 1.9E−03 | 1.0E−02 | 4.5E−04 |
| Mean | 1.0E−04 | 1.5E−03 | 2.1E−04 | 6.9E−05 | 7.1E−04 | 1.5E−03 | 1.9E−04 |
| SD | 1.2E−04 | 2.0E−03 | 3.2E−04 | 1.3E−04 | 5.8E−04 | 2.1E−03 | 1.8E−04 |
| SE | 3.6E−05 | 3.1E−04 | 7.1E−05 | 3.2E−05 | 1.9E−04 | 4.2E−04 | 7.0E−05 |
| Lower 95% CI of mean | 2.3E−05 | 9.1E−04 | 6.7E−05 | 2.5E−06 | 2.6E−04 | 6.1E−04 | 2.0E−05 |
| Upper 95% CI of mean | 1.8E−04 | 2.2E−03 | 3.6E−04 | 1.4E−04 | 1.2E−03 | 2.3E−03 | 3.6E−04 |
| Sum | 1.1E−03 | 6.4E−02 | 4.3E−03 | 1.2E−03 | 6.4E−03 | 3.8E−03 | 1.3E−03 |

TABLE 3C
Cytokine mRNA levels

| IL5RA | Healthy | Active EE | Intermediate EE | Inactive EE | Nonallergic | Allergic | CE |
|---|---|---|---|---|---|---|---|
| Minimum | 1.2E−14 | 1.7E−13 | 3.2E−18 | 8.8E−15 | 1.7E−13 | 2.3E−13 | 5.4E−14 |
| 25% Percentile | 3.8E−14 | 1.1E−12 | 4.8E−13 | 1.2E−13 | 1.1E−12 | 8.9E−13 | 1.5E−13 |
| Median | 1.6E−13 | 3.5E−12 | 1.8E−12 | 1.9E−13 | 1.8E−12 | 2.9E−12 | 4.7E−13 |
| 75% Percentile | 2.8E−13 | 8.5E−12 | 5.6E−12 | 6.8E−13 | 5.4E−12 | 8.0E−12 | 7.9E−13 |
| Maximum | 3.0E−13 | 7.7E−10 | 5.0E−11 | 4.1E−11 | 1.3E−11 | 4.6E−10 | 8.9E−13 |
| Mean | 1.6E−13 | 3.5E−11 | 5.3E−12 | 3.4E−12 | 3.6E−12 | 2.5E−11 | 4.7E−13 |
| SD | 1.2E−13 | 1.2E−10 | 1.0E−11 | 9.4E−12 | 3.9E−12 | 8.3E−11 | 3.4E−13 |
| SE | 5.4E−14 | 1.5E−11 | 2.0E−12 | 2.1E−12 | 1.3E−12 | 1.5E−11 | 1.7E−13 |
| Lower 95% CI of mean | 5.8E−15 | 4.5E−12 | 1.2E−12 | −9.9E−13 | 6.6E−13 | −5.1E−12 | −7.3E−14 |
| Upper 95% CI of mean | 3.1E−13 | 6.6E−11 | 9.5E−12 | 7.8E−12 | 6.6E−12 | 5.5E−11 | 1.0E−12 |
| Sum | 7.8E−13 | 2.1E−09 | 1.4E−10 | 6.8E−11 | 3.3E−11 | 7.8E−10 | 1.9E−12 |

### Example 4

### IL4 and IL5 Esophageal mRNA Levels as a Function of Allergy Status

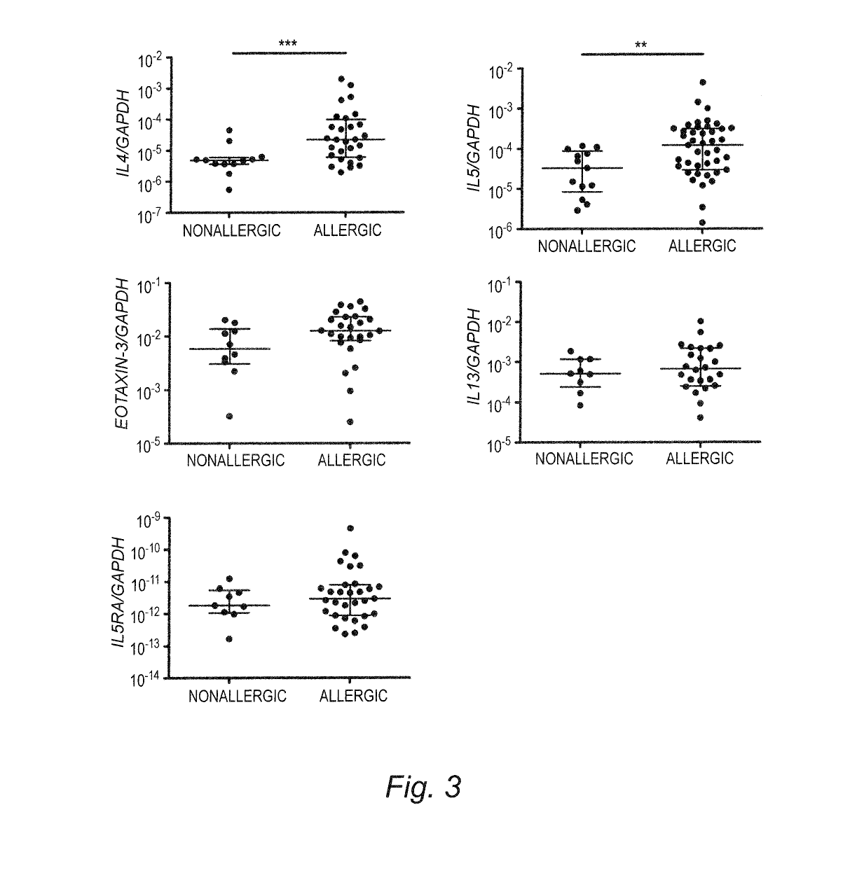

T_{H}2 cytokine levels in patients with active EE (>24 eosinophils/hpf) were measured to determine their correlation with presence of allergic disease (as determined by medical history or current diagnosis). IL4 and IL5 had significantly increased mRNA levels in patients with allergy and EE compared with patients without allergy with EE (median, 2.2×10⁻⁵; 25-75 interquartile, 6.0×10⁻⁶−9.8×10⁻⁵ vs median, 4.8×10⁻⁶; 25-75 interquartile, 3.6×10⁻⁶−5.9×10⁻⁶; P<0.0005; and median, 1.2×10⁻⁴; 25-75 interquartile, 2.9×10⁻⁵−3.1×10⁻⁴ vs median, 3.3×10⁻⁵; 25-75 interquartile, 8.3×10⁻⁶−8.7×10⁻⁵; P<0.005, respectively). No significant changes in eotaxin-3 or IL13 levels (FIG. 3) were found in patients with EE with and without allergy (median, 1.3×10⁻²; 25-75 interquartile, 8.3×10⁻³−2.3×10⁻² vs median, 5.8×10⁻³; 25-75 interquartile, 3.0×10⁻³−1.4×10⁻² and median, 6.7×10⁻⁴; 25-75 interquartile, 2.5×10⁻⁴−2.1×10⁻³ vs median, 5.1×10⁻⁴; 25-75 interquartile, 2.4×10⁻⁴−2.1×10⁻³, respectively; FIG. 3). No significant change in IL2 mRNA was observed as a function of the allergy history. These results indicate that IL4 and IL5 are dysregulated in patients with allergy and EE compared with patients without allergy with EE and reflect the systemic allergic history of the patients rather than the activity of the disease.

### Example 5

### Correlation of Cytokine Expression in Patients with Active EE

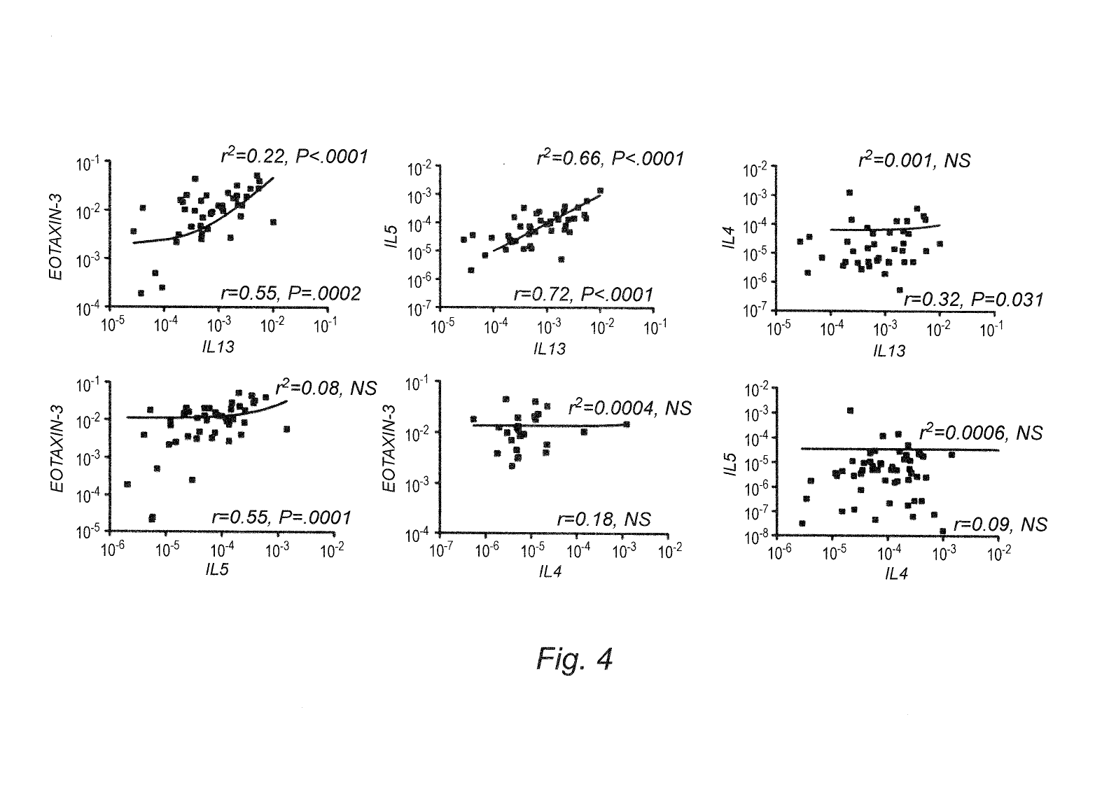

Cytokine levels were measured to determine whether abnormal cytokine levels would correlate with each other in patients with active EE. The correlation between IL13 and other T_{H}2 cytokines as well as eotaxin-3 was studied because IL13 has been shown to induce the latter cytokine A significant Spearman correlation was found between IL13 and eotaxin-3 (r, 0.55; P=0.0002) and between IL5 and eotaxin-3 (r, 0.55; P=0.0001), and a surprisingly high correlation was found between IL13 and IL5 (r, 0.72; P<0.0001; FIG. 4). A weak correlation was found between the expression of IL13 and IL4 (r, 0.32; P<0.05), and no significant correlation was found between IL4 and eotaxin-3 (r, 0.18; P>0.05) or between IL4 and IL5 (r, 0.09; P>0.05). Linear regression analysis was significant when comparing IL13 with either eotaxin-3 or IL5. No significant linear regressions were seen between IL4 and IL13, IL5 and eotaxin-3, IL4 and eotaxin-3, or IL4 and IL5. IL5RA mRNA levels showed no correlation with any of the cytokine mRNA quantified and also showed no correlation with eosinophil number in the active EE group (r, −0.0229; P=0.85; see Table 4). These results indicate that IL13 mRNA expression is highly correlated to IL5 and eotaxin-3 expression.

TABLE 4
Spearman (rank) correlation of cytokines and cytokine receptor with
eosinophil levels

| | Correlation | | | |
|---|---|---|---|---|
| | coefficient | | Correlation coefficient | |
| Genes | in active EE | P value | in the whole cohort | P value |
| IL13 | 0.442 | .00397 | 0.716 | <.0001 |
| Eotaxin-3 | 0.473 | .0190 | 0.767 | <.0001 |
| IL4 | 0.155 | .262 | 0.412 | <.0001 |
| IL5 | 0.0656 | .605 | 0.470 | <.0001 |
| IL5RA | −0.0229 | .85 | 0.460 | <.0001 |

### Example 6

### Blood Cytokine Levels in Patients with EE

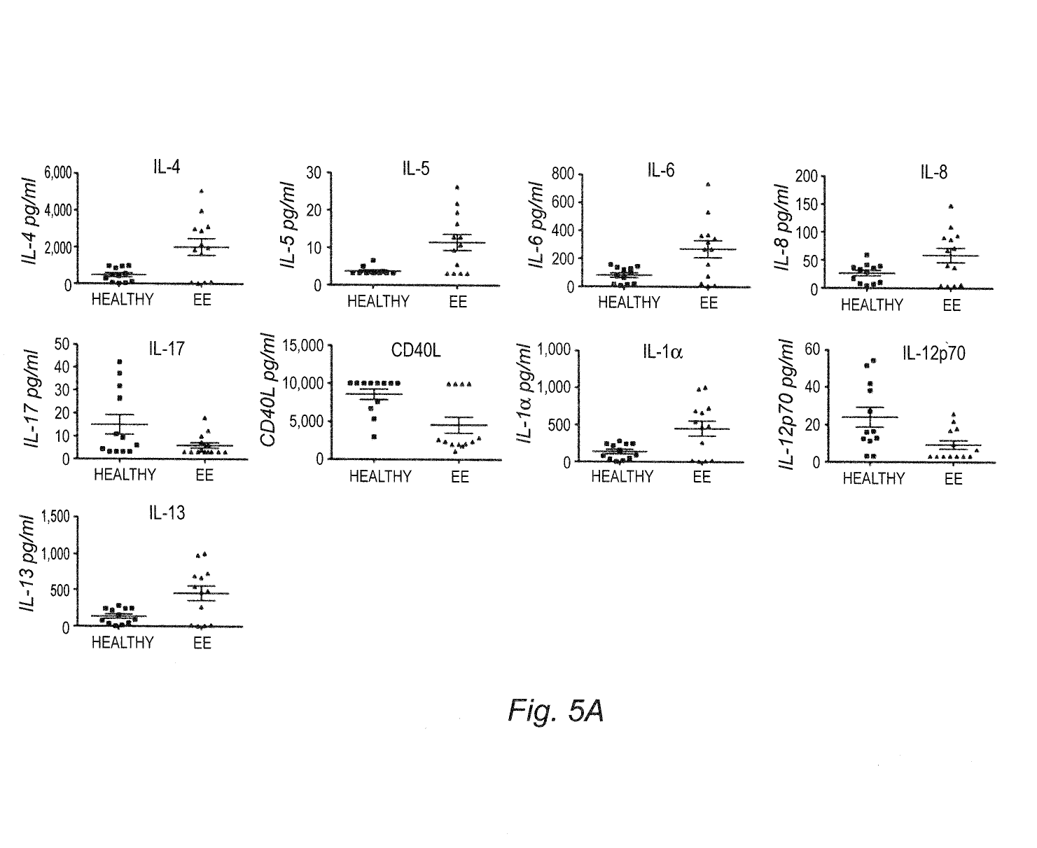

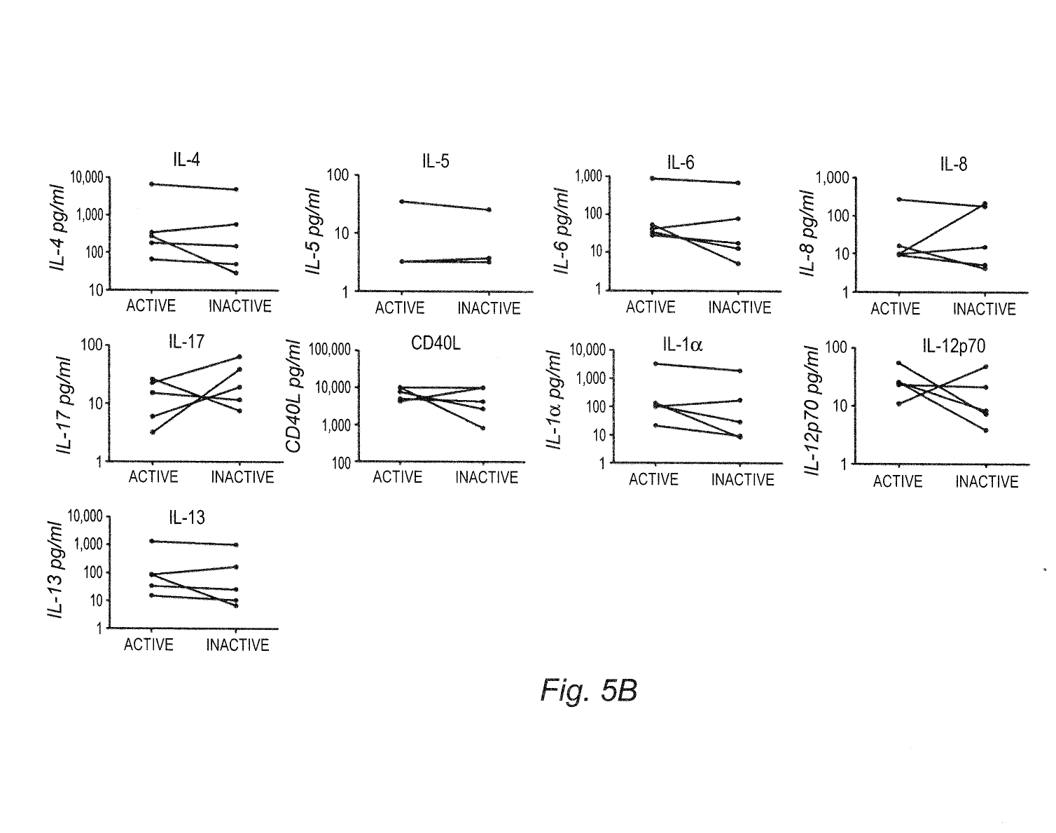

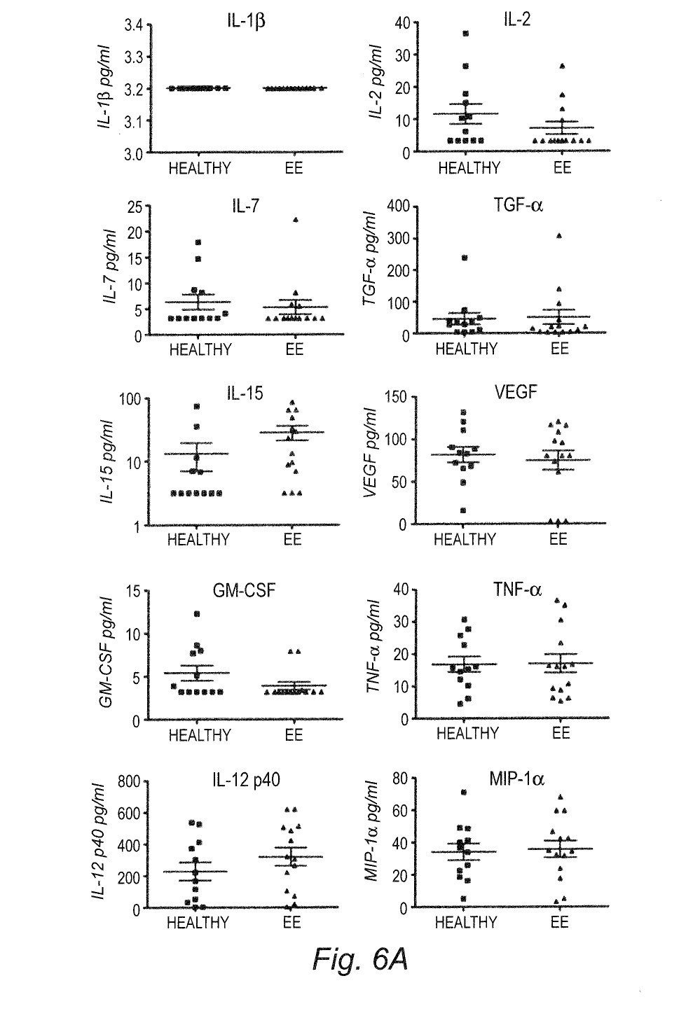

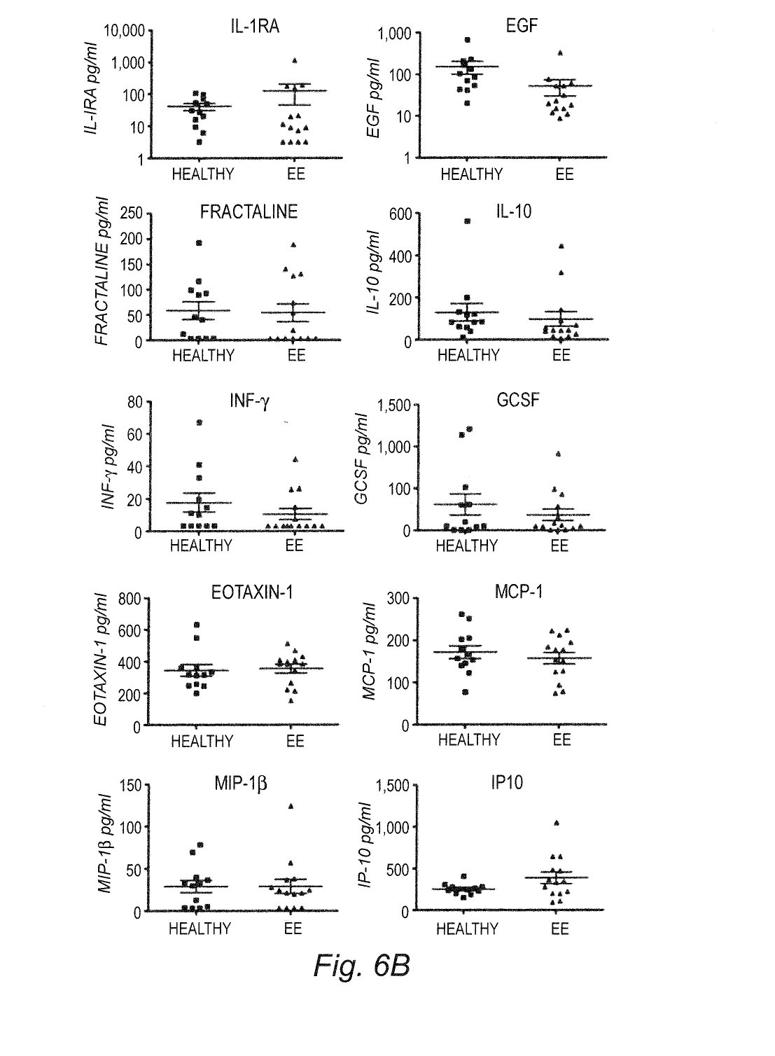

Systemic levels of cytokines were measured to determine whether such levels were abnormal in EE. Cytokine levels of non-EE (healthy, n=12) and active EE patients (EE, n=13; FIGS. 5A, 5B, 6A, 6B, and 7A-D); were quantified using a human cytokine panel multiplex assay containing 84 cytokines IL-13, IL-4, IL-6, IL-5, CD40 ligand, IL-12p70, and epidermal growth factor (EGF) were significantly modified in EE compared with healthy subjects (FIG. 5A) and allowed discrimination of the patient diagnosis with 100% sensitivity and specificity. Cytokines were also quantified in 5 patients in active and inactive stages of the disease, and no difference in the average or paired analysis was observed between active and inactive EE, even for cytokine levels that were significantly different in healthy subjects versus EE patients (FIG. 5B). In this learning set of patients, cytokine levels were significantly decreased for IL-10, IL-1Rα, and vascular endothelial growth factor (VEGF). These results indicate that the activity of the disease does not consistently affect these systemic cytokine levels.

### Example 7

### Use of Blood Cytokine Levels as a Predictive Diagnostic Tool for Ee

The scoring panel designed for the learning set (Table 1) was used to predict diagnosis of prospectively recruited patients. Blind blood plasma samples from 36 patients underwent analysis (Tables 5A-D). Of the 36 subjects tested, the scoring system identified 14 potential patients with EE; 22 samples were predicted to belong to patients without EE (Table 6). After the diagnosis was revealed and linked with the data, 3 of the 14 positive patients were patients without EE, indicating a 79% positive predictive value. Out of the 22 patients predicted to be patients without EE, 15 were truly negative, indicating a 68% negative predictive value. The specificity of the test was 83%, with 3 false-positives, for the 18 patients without EE that were tested. In conjunction, 7 of the 18 patients with EE were not identified by the test, demonstrating 61% sensitivity (Table 7). The test was also able to diagnose the presence of allergy (as determined by medical history or current diagnosis) among all the patients, regardless of the esophageal diagnosis, with a 78% positive predictive value, 32% negative predictive value, 70% specificity, and 42% sensitivity. No significant differences were observed for most cytokines between healthy subjects and patients with EE.

TABLE 5A
Cytokine levels in 36 patients

| Sample ID # | EGF | Eotaxin | FGF-2 | FLt-3 L | Fractalkine | G-CSF | GM-CSF | GRO | IFNα2 | IFN-γ |
|---|---|---|---|---|---|---|---|---|---|---|
| 1 | 82 | 180 | 67 | ND | 131 | ND | 155 | 993 | 910 | 5 |
| 2 | 30 | 273 | 29 | ND | 252 | ND | 169 | 1344 | 1701 | 9 |
| 3 | 161 | 273 | 109 | 32 | 949 | 118 | 458 | 1801 | 2614 | 21 |
| 4 | 66 | 178 | 49 | ND | 62 | 34 | 195 | 769 | 1537 | 25 |
| 5 | 40 | 293 | 61 | ND | 215 | 29 | 250 | 752 | 1557 | 15 |
| 6 | 187 | 215 | 57 | 36 | 302 | ND | 618 | 960 | ND | 30 |
| 7 | 64 | 235/321* | 45 | ND | 97 | ND | 209 | 711 | ND | 11 |
| 8 | 99 | 165 | 63 | ND | 133 | ND | 252 | 591 | ND | 47 |
| 9 | 3 | 263 | 44 | ND | 405/210* | ND | 275 | 468 | ND | 38/18* |
| 10 | 49 | 275 | 50 | ND | 155 | ND | 246 | 796 | 504 | 17 |
| 11 | 55 | 267 | 50 | ND | 100 | ND | 206 | 786 | ND | 18 |
| 12 | 74 | 314 | 73 | 40 | 1′004 | ND | 198 | 825 | ND | 63 |
| 13 | 33 | 218 | 40 | ND | 35 | ND | 69 | 770 | 725 | 3 |
| 14 | 30 | 160 | 31 | ND | 90 | 21 | 134 | 1018 | 2374 | 9 |
| 15 | 53 | 118 | 54 | ND | 58 | ND | 92 | 764 | ND | 4 |
| 16 | 88/63* | 290 | 30 | 151 | 44 | ND | 121 | 1092 | ND | 37/22* |
| 17 | 36 | 330 | 43 | ND | 117/33* | 29 | 166 | 1289 | 3337 | 7 |
| 18 | 35 | 316 | 26 | ND | ND | ND | 59 | 849 | ND | 16 |
| 19 | 46 | 336 | 46 | 20 | 79 | 37 | 156 | 1337 | 1650 | 9 |
| 20 | 55 | 235 | 40 | 51 | 80 | ND | 153 | 804 | ND | 8 |
| 21 | 56 | 303 | 75/128* | 31 | 1656/2510* | ND | 364/688* | 1430 | ND | 93 |
| 22 | 52 | 130 | 49 | 23 | 49 | ND | 109 | 775 | ND | 9 |
| 23 | 41 | 205 | 30/41* | ND | 39/113* | ND | 110 | 175/244* | ND | 5 |
| 24 | 107 | 327 | 41 | ND | 166 | ND | 200 | 548 | 578 | 28 |
| 25 | ND | 202/323* | 26/41* | ND/22* | 164 | ND | 79 | 394/520* | 515 | 9/23* |
| 26 | 31 | 272 | 26 | 52 | 31 | ND | 63 | 955 | ND | 6 |
| 27 | 112 | 140/220* | 72/100* | ND/31* | 162 | ND | 364/613* | 1573 | 1193 | 15/31* |
| 28 | 140 | 256 | 161 | 178 | 1687 | 162 | 637 | 808 | 1684 | 50 |
| 29 | ND | 124 | 28 | ND | ND | ND | 103 | 976 | ND | 6 |
| 30 | 47 | 164 | 41 | ND | ND | ND | 113 | 1234 | 1336 | 59 |
| 31 | 51 | 165 | 44 | ND | 106 | ND | 146 | 581 | 1637 | ND |
| 32 | 83 | 294 | 84 | 114 | 807 | ND | 201 | 961 | ND | 25 |
| 33 | ND | 213 | 33 | 23 | 22 | ND | 124 | 319 | ND | 11 |
| 34 | 187 | 394 | 189 | 133 | 1288 | 182 | 1279 | 1289 | 4342 | 76 |
| 35 | 67 | 193 | 44 | ND | ND | † | 102 | 1217 | ND | 5 |
| 36 | 24 | 234 | 37 | 63 | 313 | ND | 94 | 443 | 698 | 23 |

ND, Not detected;
OOR, out of range.
These data were obtained by using “Research Only” kits. The data cannot be used for clinical or diagnostic purposes.
*High coefficient of variation (CV), both replicates reported.
† No result because of insufficient bead count.

TABLE 5B
Cytokine levels in 36 patients

| Sample ID # | IL-1α | IL-1β | IL-1RA | IL-2 | IL-3 | IL-4 | IL-5 | IL-7 | IL-8 | IL-9 |
|---|---|---|---|---|---|---|---|---|---|---|
| 1 | 394 | 4 | 47 | 8 | 4 | 165 | 4 | 97 | 17 | ND |
| 2 | 296 | 23 | 153 | 4 | 8 | 300 | 10 | 115 | 39 | 70 |
| 3 | 692 | 8 | 59 | 18 | 4 | 812 | 17 | † | 57 | 26 |
| 4 | 157 | 5 | 52 | 52 | 7 | 133 | 9 | 195 | 64 | ND |
| 5 | 282 | 11 | 108 | 6 | 12 | 230 | 9 | 201 | 43 | 41 |
| 6 | 528 | 7 | 31 | 20 | ND | ND | ND | ND | 36 | ND |
| 7 | 241 | ND | 50 | 10/6* | ND | ND | ND | ND | 16 | ND |
| 8 | 181 | ND | 32 | 14 | ND | ND | ND | ND | 38 | ND |
| 9 | 439 | ND | 48 | 11 | ND | ND | ND | ND | 38/15* | ND |
| 10 | 222 | ND | 41 | 12 | ND | ND | ND | ND | 12 | ND |
| 11 | 211 | ND | 41 | 13 | ND | ND | ND | † | 19 | ND |
| 12 | 224 | ND | 39 | 22 | 13 | 35 | ND | ND | 34 | ND |
| 13 | 142 | ND | 25 | 4 | ND | 45 | ND | 44 | 14 | ND |
| 14 | 204 | 13 | 123 | ND | 4 | 415 | 14 | 258 | 57 | 58 |
| 15 | 221 | ND | 29 | 9 | ND | ND | ND | ND | 5 | ND |
| 16 | 167 | ND | 24 | ND | ND | ND | ND | ND | 27/16* | ND |
| 17 | 111 | 6 | 61 | 7 | ND | 612 | 13 | 255 | 81 | 20 |
| 18 | 143 | ND | ND | ND | ND | ND | ND | ND | 10 | ND |
| 19 | 196 | 14 | 87 | 4 | 4 | 450 | 9 | 245 | 51 | 38 |
| 20 | 438 | ND | ND | 4 | 3 | ND | ND | † | 6 | ND |
| 21 | 375 | 17/67* | 39/84* | 8/26* | ND | ND | ND | † | 49 | 324 |
| 22 | 153 | 10 | 85 | 4 | 7 | ND | 4 | 29 | 8 | 51 |
| 23 | 147/227* | 21 | 80 | 3/7* | 7 | ND | 5 | 56/84* | 10 | 31 |
| 24 | 343 | 33 | 215 | 29 | 13 | ND | 12 | † | 35 | 400 |
| 25 | 83 | 14/41* | 65 | 5/13* | ND | 27/92* | 4 | † | 13/26* | 38 |
| 26 | 247 | ND | ND | ND | ND | ND | ND | † | 7 | ND |
| 27 | 311 | 6 | 59 | 20 | ND | 87/170* | 5 | 120/171* | 25 | ND |
| 28 | 365 | 60 | 363 | 83 | 161 | 20 | 29 | 104 | 22 | 162 |
| 29 | 260 | ND | 98 | ND | ND | ND | ND | ND | 4 | ND |
| 30 | 300 | ND | ND | ND | ND | 52 | 5 | 152 | 33 | ND |
| 31 | 408 | 9 | 93 | 5 | 10 | 198 | 11 | 288 | 40 | 32 |
| 32 | 690 | ND | 47 | 26 | 15 | ND | 11 | ND | 8 | ND |
| 33 | 148 | ND | ND | 4 | ND | ND | ND | ND | 9 | ND |
| 34 | 539 | 57 | 322 | 83 | 56 | 1484 | 35 | 457 | 94 | 170 |
| 35 | 160 | ND | 73 | ND | ND | ND | ND | ND | 9 | ND |
| 36 | 147 | ND | 28 | 16/10* | ND | 9 | ND | 80 | 9 | ND |

ND, Not detected;
OOR, out of range.
These data were obtained by using “Research Only” kits. The data cannot be used for clinical or diagnostic purposes.
*High coefficient of variation (CV), both replicates reported.
† No result because of insufficient bead count.

TABLE 5C
Cytokine levels in 36 patients

| Sample ID # | IL-10 | IL-12(p40) | IL-12(p70) | IL-13 | IL-15 | IL-17 | IP-10 | MCP-1 | MCP-3 | MDC |
|---|---|---|---|---|---|---|---|---|---|---|
| 1 | 12 | 72 | 5 | 7 | 10 | ND | 532 | 336 | 80 | 1418 |
| 2 | 47 | 275 | ND | 25 | 50 | 4 | 404 | 404 | 163 | 2699 |
| 3 | 58 | 132 | 9 | 13 | 29 | 4 | 1372 | 383 | 227 | 4096 |
| 4 | 9 | 63 | 10 | 9 | 16 | 35 | 236 | 318 | 106 | 2997 |
| 5 | 36 | 377 | 29 | 26 | 33 | 4 | 418 | 393 | 102 | 1775 |
| 6 | 4 | 165 | 49 | 140 | 6 | 47 | 390 | 186 | ND | 3193 |
| 7 | 4 | 93 | 8 | 30 | 4 | 21 | 275 | 359 | ND | 2058 |
| 8 | 10 | 176 | 25 | 238 | 4 | 69 | 288 | 328 | ND | 1206 |
| 9 | 4 | ND | 148/60* | 204/108* | ND | 74/19* | 368 | 371 | ND | 1857/845* |
| 10 | 4 | 198 | 12 | 14 | 4 | 15 | 273 | 352 | 18 | 1799 |
| 11 | 7 | 96 | 11 | 15 | 7 | 17 | 259 | 339 | 20/29* | 2262 |
| 12 | 26 | 47 | 92 | ND | 6 | 51 | 317 | 343 | 42 | 3008 |
| 13 | ND | 234 | ND | ND | 7 | ND | 223 | 282 | 70 | 846 |
| 14 | 21 | 293 | 7 | 28 | 61 | 30 | 333 | 309 | 156 | 1123 |
| 15 | 8 | ND | ND | ND | ND | ND | 326 | 350 | 17 | 747 |
| 16 | ND | 237 | 84 | ND | ND | 41/21* | 273 | 290 | ND | 2368 |
| 17 | 15 | 99 | 12 | 6 | 23 | 7 | 212 | 362 | 238 | 2682 |
| 18 | ND | ND | ND | 14 | ND | 3 | 188 | 200 | 26 | 1536 |
| 19 | 33 | 350 | 6 | 25 | 36 | 8 | 278 | 422 | 143 | 2014 |
| 20 | ND | ND | 14 | ND | ND | 8 | 568 | 260 | ND | 1564 |
| 21 | 4 | 213 | 8/16* | 25 | 10 | 67 | 315 | 274 | 25 | 2263 |
| 22 | 17 | 377 | 6 | 14 | 31 | ND | 200 | 335 | 18 | 1798 |
| 23 | 16 | 141 | 4 | 21 | 31 | 8 | 235 | 273 | 24 | 3031 |
| 24 | 58 | 535 | 47 | 56 | 70 | 25 | 366 | 410 | 33 | 908 |
| 25 | 21 | 221 | 5/13* | 22 | 22 | 8/26* | 93/139* | 197/355* | 52/74* | 786/1704* |
| 26 | ND | 82 | ND | ND | ND | 11 | 416 | 499 | ND | 667 |
| 27 | 22 | 169 | 10 | 7 | 22 | 8 | 521 | 473 | 134/213* | 1746 |
| 28 | 118 | 993 | 134 | 128 | 131 | 46 | 549 | 297 | 79 | 2077 |
| 29 | 31 | 117 | ND | ND | ND | ND | 433 | 348 | ND | 2351 |
| 30 | 6 | 203 | ND | ND | 7 | 25 | 333 | 481 | 79 | 1885 |
| 31 | 20 | 225 | 3 | 23 | 30 | 5 | 728 | 181 | 103 | 779 |
| 32 | 5 | 73 | 50 | 15 | 4 | 12 | 945 | 275 | 29 | 1311 |
| 33 | ND | ND | 10 | 7 | ND | 37 | 251 | 292 | ND | 1494 |
| 34 | 272 | 1097 | 164 | 108 | 154 | 80 | 600 | 340 | 260 | 1535 |
| 35 | ND | ND | ND | ND | ND | ND | 280 | 337 | ND | 3676 |
| 36 | ND | ND | 39 | ND | 3 | 16 | 284 | 297 | 46 | 940 |

ND, Not detected;
OOR, out of range.
These data were obtained by using “Research Only” kits. The data cannot be used for clinical or diagnostic purposes.
*High coefficient of variation (CV), both replicates reported.
†No result because of insufficient bead count.

TABLE 5D
Cytokine levels in 36 patients

| Sample ID # | MIP-1α | MIP-1β | sCD40L | sIL-2Ra | TGFα | TNFα | TNFβ | VEGF | IL-6 |
|---|---|---|---|---|---|---|---|---|---|
| 1 | 39 | 42 | 9460 | 254 | 6 | 15 | 10 | 83 | 4 |
| 2 | 124 | 87 | 781 | 77 | 7 | 13 | ND | 53 | 31 |
| 3 | 109 | 70 | OOR | 414 | 203 | 20 | 6 | 290 | 20 |
| 4 | 147 | 77 | 2873 | 44 | 20 | 9 | 4 | 92 | 15 |
| 5 | 100 | 76 | 3166 | 153 | 26 | 12 | 11 | 80 | 34 |
| 6 | 152 | 107 | 2873 | 86 | 16 | 12 | 12 | 300 | 26 |
| 7 | 89 | 45 | 1890 | 49 | 9 | 8 | ND | 66 | 7 |
| 8 | 179 | 217 | 667 | 175 | 18 | 8 | ND | 276 | 92 |
| 9 | 129/86* | 103/54* | 862 | 55 | 24 | 11 | ND | 238 | 31/14* |
| 10 | 97 | 41 | 2276 | 59 | 14/6* | 9 | ND | 103 | 9 |
| 11 | 91 | 51 | 1563 | 48 | 11 | 8 | ND | 85 | 10 |
| 12 | 139 | 136 | 907 | 134 | 24 | 19 | 24 | 296 | 101 |
| 13 | 53 | 29 | 1499 | 70 | ND | 5 | ND | ND | 5 |
| 14 | 116 | 30 | 982 | ND | 59 | 7 | 12 | 90 | 21 |
| 15 | 64 | 29 | 1761 | 106 | 5 | 5 | 4 | 36 | ND |
| 16 | 91/60* | 94/63* | 1094 | 169/107* | 11/7* | 6 | ND | 213 | 53/32* |
| 17 | 72 | 44 | 993 | 87 | 102 | 10 | 4 | 155 | 15 |
| 18 | 33 | 21 | 3787 | 26 | ND | ND | ND | ND | ND |
| 19 | 98 | 41 | 1695 | 108 | 27 | 7 | ND | 79 | 22 |
| 20 | ND | 36 | 1909 | 96 | 8 | 8 | 4 | 103 | 10 |
| 21 | 143/190* | 126/181* | 2499 | 94 | 22 | 4 | ND | 149 | 21/40* |
| 22 | 87 | 30 | 4911 | 211 | ND | 7 | ND | 45 | 27 |
| 23 | 90 | 37 | 638 | 66 | 5 | 5 | 4 | 47 | 23 |
| 24 | 157 | 110 | 2291 | 153 | 21 | 9 | 4 | 111/175* | 115 |
| 25 | 64/90* | 51/119* | 518/1122* | 72 | 6/10* | 4 | 4 | 66/113* | 17 |
| 26 | 38 | 83 | 952 | ND | 4 | 5 | ND | ND | 16 |
| 27 | 156/236* | 51/76* | 1′294 | 149 | 155/359* | 14 | 3 | 406 | 11 |
| 28 | 183 | 152 | 2093 | 433 | 38 | 42 | 170 | 329 | 83 |
| 29 | 24 | 31 | 627 | 170 | ND | 7 | ND | ND | 10 |
| 30 | 120 | 63 | 2802 | 39 | 4 | 7 | ND | 74 | 20 |
| 31 | 74 | 37 | 2217 | 49 | 8 | 4 | 7 | 64 | 9 |
| 32 | 73 | 74 | 8909 | 185 | 17 | 16 | 22 | 184 | 22 |
| 33 | 46 | 21 | 391/650* | ND | 4 | 4 | ND | 162 | 17 |
| 34 | 221 | 228 | 3464 | 306 | 65 | 36 | 112 | 324 | 99 |
| 35 | ND | 23 | 7005 | 91 | ND | 5 | ND | ND | ND |
| 36 | 57 | 72 | 828 | 69 | 16 | 6 | 10 | 149 | ND |

ND, Not detected;
OOR, out of range.
These data were obtained by using “Research Only” kits. The data cannot be used for clinical or diagnostic purposes.
*High coefficient of variation (CV), both replicates reported.
†No result because of insufficient bead count.

TABLE 6
Scoring of the 36 blind samples, diagnosis, allergic history, and
peak eosinophil counts in esophageal biopsies

| Samples | IL-1α | IL-4 | L-5 | IL-6 | IL-12 p70 | L-13 | IL-17 | CD40L | Score | EE | Allergy | Eos# |
|---|---|---|---|---|---|---|---|---|---|---|---|---|
| 1 | 0 | 0 | 0 | 0 | 1 | 0 | 1 | 0 | 2 | Yes | Yes | 207 |
| 2 | 0 | 0 | 1 | 0 | 1 | 0 | 1 | 1 | 4 | Yes | No | 78 |
| 3 | 0 | 0 | 1 | 0 | 1 | 0 | 1 | 0 | 3 | Yes | No | 50 |
| 4 | 0 | 0 | 1 | 0 | 1 | 0 | 0 | 1 | 3 | Yes | No | 267 |
| 5 | 0 | 0 | 1 | 0 | 0 | 0 | 1 | 0 | 2 | Yes | Yes | 169 |
| 6 | 0 | 0 | 0 | 0 | 0 | 0 | 0 | 1 | 1 | No | Yes | 0 |
| 7 | 0 | 0 | 0 | 0 | 1 | 0 | 0 | 1 | 2 | No | No | 0 |
| 8 | 0 | 0 | 0 | 0 | 0 | 0 | 0 | 1 | 1 | No | No | 0 |
| 9 | 0 | 0 | 0 | 0 | 0 | 0 | 0 | 1 | 1 | No | No | 0 |
| 10 | 0 | 0 | 0 | 0 | 1 | 0 | 0 | 1 | 2 | No | Yes | 0 |
| 11 | 0 | 0 | 0 | 0 | 1 | 0 | 0 | 1 | 2 | No | Yes | 0 |
| 12 | 0 | 0 | 0 | 0 | 0 | 0 | 0 | 1 | 1 | No | Yes | 0 |
| 13 | 0 | 0 | 0 | 0 | 1 | 0 | 1 | 1 | 3 | Yes | Yes | 194 |
| 14 | 0 | 0 | 1 | 0 | 1 | 0 | 0 | 1 | 3 | Yes | Yes | 186 |
| 15 | 0 | 0 | 0 | 0 | 1 | 0 | 1 | 1 | 3 | Yes | Yes | 40 |
| 16 | 0 | 0 | 0 | 0 | 0 | 0 | 0 | 1 | 1 | Yes | Yes | 106 |
| 17 | 0 | 0 | 1 | 0 | 1 | 0 | 1 | 1 | 4 | Yes | Yes | 27 |
| 18 | 0 | 0 | 0 | 0 | 1 | 0 | 1 | 0 | 2 | Yes | No | 29 |
| 19 | 0 | 0 | 1 | 0 | 1 | 0 | 1 | 1 | 4 | Yes | Yes | 154 |
| 20 | 0 | 0 | 0 | 0 | 1 | 0 | 1 | 1 | 3 | Yes | Yes | 97 |
| 21 | 0 | 0 | 0 | 0 | 1 | 0 | 0 | 1 | 2 | No | No | 0 |
| 22 | 0 | 0 | 0 | 0 | 1 | 0 | 1 | 0 | 2 | Yes | Yes | 81 |
| 23 | 0 | 0 | 0 | 0 | 1 | 0 | 1 | 1 | 3 | Yes | Yes | 136 |
| 24 | 0 | 0 | 1 | 0 | 0 | 0 | 0 | 1 | 2 | Yes | Yes | 169 |
| 25 | 0 | 0 | 0 | 0 | 1 | 0 | 0 | 1 | 2 | No | Yes | 0 |
| 26 | 0 | 0 | 0 | 0 | 1 | 0 | 1 | 1 | 3 | No | Yes | 0 |
| 27 | 0 | 0 | 0 | 0 | 1 | 0 | 1 | 1 | 3 | No | Yes | 2 |
| 28 | 0 | 0 | 1 | 0 | 0 | 0 | 0 | 1 | 2 | No | Yes | 0 |
| 29 | 0 | 0 | 0 | 0 | 1 | 0 | 1 | 1 | 3 | Yes | Yes | 105 |
| 30 | 0 | 0 | 0 | 0 | 1 | 0 | 0 | 1 | 2 | No | No | 0 |
| 31 | 0 | 0 | 1 | 0 | 1 | 0 | 1 | 1 | 4 | No | Yes | 0 |
| 32 | 0 | 0 | 1 | 0 | 0 | 0 | 1 | 0 | 2 | Yes | Yes | 103 |
| 33 | 0 | 0 | 0 | 0 | 1 | 0 | 0 | 1 | 2 | No | Yes | 0 |
| 34 | 0 | 1 | 1 | 0 | 0 | 0 | 0 | 0 | 2 | No | Yes | 0 |
| 35 | 0 | 0 | 0 | 0 | 1 | 0 | 1 | 0 | 2 | No | No | 0 |
| 36 | 0 | 0 | 0 | 0 | 0 | 0 | 0 | 1 | 1 | No | Yes | 0 |

Eos#, Maximum eosinophil count/hpf.

TABLE 7
Assessment of the specificity and sensitivity of the test

| | | EE diagnosis | | |
|---|---|---|---|---|
| | | Positive | Negative | |
| | | n = 18 | n = 18 | |
| Test | Positive | TP = 11 | FP = 3 | PPV = 79% |
| results | n = 14 | FN = 7 | TN = 15 | NPV = 68% |
| | Negative | Sensitivity = 61% | Specificity = 83% | |
| | n = 22 | | | |

FN, False negative;
FP, false positive;
NPV, negative predictive value, TN/(FN + TN);
PPV, positive predictive value, TP/(TP + FP);
TN, true negative;
TP, true positive.
Sensitivity = TP/(TP + FN).
Specificity = TN/(TN + FP).

The various methods and techniques described above provide a number of ways to carry out embodiments of the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that can have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the invention disclosed herein are illustrative of the principles of the embodiments of the invention. Other modifications that can be employed can be within the scope of the invention. Thus, by way of example, but not of limitation, alternative configurations of the embodiments of the invention can be utilized in accordance with the teachings herein. Accordingly, embodiments of the present invention are not limited to that precisely as shown and described.

What is claimed is:

1. A method of providing or enhancing a diagnosis of EE, comprising:
obtaining a sample from a patient having at least one indication of EE;
quantifying from the sample an amount of at least one analyte, wherein the analyte is: any of the cytokines listed in Table 1, any of the cytokines listed in Table 2, or an mRNA corresponding to any member of the group or its receptor, wherein an altered level of the at least one analyte correlates with a positive diagnosis of EE;
providing or enhancing a diagnosis of EE, based upon the quantifying step.

2. The method of claim 1, wherein the at least one analyte is: any of the cytokines listed in Table 1, or an mRNA corresponding to any member of the group or its receptor.

3. The method of claim 1, wherein at least two analytes are quantified.

4. The method of claim 1, wherein at least four analytes are quantified.

5. The method of claim 1, wherein all of the cytokines listed in Table 2, or an mRNA corresponding to any member of the group or its receptor are quantified.

6. The method of claim 2, wherein all of the cytokines listed in Table 1, or an mRNA corresponding to any member of the group or its receptor are quantified.

7. The method of claim 1, wherein the sample is an esophageal biopsy.

8. The method of claim 1, wherein the sample comprises esophageal mucosa.

9. The method of claim 1, wherein the sample is blood.

10. The method of claim 1, wherein the indication of EE comprises at least one of a gastrointestinal complaint and esophageal eosinophil infiltration.

11. The method of claim 10, wherein the gastrointestinal complaint comprises at least one of: failure to thrive, vomiting, abdominal pain, dysphagia, and food impaction.

12. The method of claim 1, wherein the diagnosis of EE is classified as allergic, non-allergic, active, intermediate, or inactive EE, or a variable degree of disease activity.

13. The method of claim 12, wherein the EE diagnosis classification is used to predict the patient's level of response to a selected therapy.

14. The method of claim 13, wherein the selected therapy is at least one of: allergen removal, steroid treatment, dietary management, or the use of proton pump inhibitors (PPIs), topical glucocorticoids, humanized antibodies against relevant cytokines, small molecule inhibitors of an eosinophil, small molecule inhibitors of an allergic disease activation pathway, and any combination thereof.

15. The method of claim 1, wherein the diagnosis of EE is enhanced by combining information from the quantifying step with one or more other tests for or indicia of EE.

16. The method of claim 12, wherein the other tests for or indicia of EE are selected from the group consisting of: determination of allergic status, quantification of biomarkers associated with allergic status, determination of atopic status, quantification of biomarkers associated with atopic status, endoscopy with biopsy analysis, detection of eosinophils, detection of eotaxin-3, detection of eosinophil-derived neurotoxin, and detection of IL-5 protein.

17. A diagnostic kit, test, or array, comprising materials for quantification of at least two analytes, wherein the at least two analytes are: any of the cytokines listed in Table 1, any of the cytokines listed in Table 2, or an mRNA corresponding to any member of the group or its receptor, or any combinations thereof.

18. The diagnostic kit, test, or array of claim 17, wherein the at least two analytes are: any of the cytokines listed in Table 1, or an mRNA corresponding to any member of the group or its receptor, or any combination thereof.
